SEQUENCE LISTING (10 sequences)

SEQ ID NO: 1 - protein, 117 aa, Artificial Sequence [sequence omitted]

SEQ ID NO: 2 - protein, 107 aa, Artificial Sequence [sequence omitted]

SEQ ID NO: 3 - protein, 8 aa, Artificial Sequence
  HCDR1
GFTFSNFG

SEQ ID NO: 4 - protein, 8 aa, Artificial Sequence
  HCDR2
ISGGGRDT

SEQ ID NO: 5 - protein, 10 aa, Artificial Sequence
  HCDR3
VKWGNIYFDY

SEQ ID NO: 6 - protein, 6 aa, Artificial Sequence
  LCDR1
LSINTF

SEQ ID NO: 7 - protein, 3 aa, Artificial Sequence
  LCDR2
AAS

SEQ ID NO: 8 - protein, 9 aa, Artificial Sequence
  LCDR3
QQSSNTPFT

SEQ ID NO: 9 - protein, 444 aa, Artificial Sequence
  HC
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  LC
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0270321 A1
(43) Pub. Date: Aug. 28, 2025

(54) METHODS OF TREATING LUNG CANCER BY ADMINISTERING A PD-1 INHIBITOR

(71) Applicant: Regeneron Pharmaceuticals, Inc., Tarrytown, NY (US)

(72) Inventors: Petra Rietschel, Tarrytown, NY (US); Israel Lowy, Dobbs Ferry, NY (US)

(21) Appl. No.: 18/547,192

(22) PCT Filed: Feb. 22, 2022
(86) PCT No.: PCT/US2022/017247

§ 371 Date: Aug. 21, 2023

Related U.S. Application Data
(60) Provisional application No. 63/276,006, filed on Nov. 5, 2021.
(60) Provisional application No. 63/238,309, filed on Aug. 30, 2021.
(60) Provisional application No. 63/182,454, filed on Apr. 30, 2021.
(60) Provisional application No. 63/174,480, filed on Apr. 13, 2021.
(60) Provisional application No. 63/152,608, filed on Feb. 23, 2021.

(51) Int. Cl. C07K 16/28; A61K 39/00; A61K 45/06; A61P 35/04

(52) U.S. Cl. CPC C07K 16/2818; A61K 45/06; A61P 35/04; A61K 2039/505; C07K 2317/21; C07K 2317/565

(57) ABSTRACT

The present disclosure provides methods for treating or inhibiting the growth of a tumor, wherein the method includes selecting a patient with lung cancer and brain metastasis in need thereof and administering to the patient a therapeutically effective amount of a programmed death 1 (PD-1) inhibitor (e.g., cemiplimab or a bioequivalent thereof). In certain embodiments, the lung cancer is non-small cell lung cancer (NSCLC) and PD-L1 is expressed in ≥50% of tumor cells. In certain embodiments, the methods lead to improvements in functioning and quality of life.

### CROSS-REFERENCE TO RELATED APPLICATIONS

This application is a U.S. national phase application under 35 U.S.C. § 371 of International Patent Application No. PCT/US2022/017247 filed Feb. 22, 2022, which claims the benefit of priority to U.S. Provisional Patent Application No. 63/276,006 filed Nov. 5, 2021, U.S. Provisional Patent Application No. 63/238,309 filed Aug. 30, 2021, U.S. Provisional Patent Application No. 63/182,454 filed Apr. 30, 2021, U.S. Provisional Patent Application No. 63/174,480 filed Apr. 13, 2021, and U.S. Provisional Patent Application No. 63/152,608 filed Feb. 23, 2021, the disclosures of all of which are hereby incorporated by reference in their entireties.

### REFERENCE TO A SEQUENCE LISTING

The sequence listing of the present application is submitted electronically as a computer readable sequence listing in ASCII format with a file name of “SeqList10926-ST25.txt,” a creation date of Jan. 25, 2024, and a size of 8,907 bytes. This sequence listing submitted is part of the specification and is hereby incorporated by reference in its entirety.

### FIELD

The present disclosure relates to methods of treating or inhibiting the growth of a tumor, including selecting a patient with lung cancer in need thereof and administering to the patient a therapeutically effective amount of a programmed death 1 (PD-1) inhibitor (e.g., cemiplimab or a bioequivalent thereof), wherein the patient has brain mestastasis.

### BACKGROUND

Lung cancer is one of the most commonly diagnosed cancers, and the leading cause of cancer-related mortality worldwide (Siegel 2016). In 2020, an estimated 228,820 new cases of lung cancer were to be diagnosed in the United States, and 135,720 people were estimated to die from the disease (American Cancer Society 2020). Non-small cell lung cancer (NSCLC) accounts for 80-85% of all lung cancers, and is composed of several histopathological subtypes, the most common of which are adenocarcinoma (40-60%) and squamous cell carcinoma (30%). The majority of patients with NSCLC are often found to have advanced cancer at the time of diagnosis.

Systemic therapy with platinum-based doublet regimens, with or without maintenance therapy was until recently the standard first-line treatment for patients with advanced NSCLC whose tumors do not have an epidermal growth factor receptor (EGFR) mutation, an anaplastic lymphoma kinase (ALK) mutation, or a c-ros oncogene 1 receptor tyrosine kinase (ROS1) fusion (Besse 2014, Ettinger 2016, Reck 2014). This is still the standard of care for patients in countries where therapies with immune checkpoint inhibitors are not approved or available. Chemotherapy for metastatic NSCLC is not curative and despite optimal treatment, patients have a median overall survival (OS) of approximately 8 to 12 months, and a 5-year survival rate of approximately 18% (Siegel, 2016). Therefore, newer therapeutic approaches are needed that will improve long-term survival and quality of life (QOL) in patients with lung cancer, particularly advanced NSCLC.

It is recognized that a complex cross-talk between cancer cells and the host immune system exists that can both inhibit and enhance tumor growth (Vinay 2015). Cancer modulates and evades the host immune response through a number of mechanisms, including formation of an immune-suppressive environment within the tumor. Programmed cell death-1 (PD-1) is an important co-receptor expressed on the surface of activated T-cells that mediates immunosuppression. The binding of PD-1 to one of its ligands, programmed cell death ligand-1 (PD-L1) or programmed cell death ligand-2 (PD-L2), results in inhibition of a cytotoxic T-cell response. Increased expression of PD-L1 in the tumor microenvironment facilitates escape from the immune-surveillance mechanism (T-cell-induced anti-tumor activity). In contrast, blockade of this interaction by using immune checkpoint inhibitors results in an enhanced T-cell response with anti-tumor activity.

Brain metastases are a common complication in many types of cancers, and are especially prevalent among lung cancer patients. As many as 40% of lung cancer patients will develop brain tumors, and more brain metastases start out as lung tumors than any other type of cancer. Brain metastases are found in approximately 10% of patients with newly diagnosed NSCLC, and 26% of patients with Stage IV NSCLC (Waqar. 2018). For patients with NSCLC and brain metastases, prognosis is generally poor with a median overall survival (OS) of 7.8 months (Ali, 2013). Local therapies such as whole brain radiotherapy have been the mainstay of treatment for brain metastases in patients with NSCLC (Chamberlain, 2017). Even if radiotherapy is associated with good local control, the response is not durable. Many patients with large-volume systemic disease cannot tolerate a delay in systemic therapies in order to undergo local therapies (Goldberg, 2020; Goldberg, 2016). Thus, there is a significant need for a safe and effective therapy for treating patients with lung cancer, such as NSCLC, and brain metastases.

### SUMMARY

In one aspect, the disclosed technology relates to a method of treating or inhibiting the growth of a tumor, including: (a) selecting a patient with lung cancer and brain metastasis; and (b) administering to the patient a therapeutically effective amount of a programmed death-1 (PD-1) inhibitor, wherein the PD-1 inhibitor is an antibody that binds specifically to PD-1 and includes three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof. In some embodiments, the lung cancer is non-small cell lung cancer. In some embodiments, the lung cancer is locally advanced or metastatic non-small cell lung cancer. In some embodiments, the lung cancer is locally advanced non-small cell lung cancer. In some embodiments, the patient is not a candidate for surgical resection or definitive chemoradiation. In some embodiments, the lung cancer is metastatic. In some embodiments, the patient has squamous or non-squamous lung cancer. In some embodiments, tumor tissue in the patient expresses PD-L1 in ≥50% of tumor cells. In some embodiments, the brain metastasis is treated, stable brain metastasis. In some embodiments, the patient has no EGFR, ALK or ROS1 aberrations.

In some embodiments, the anti-PD-1 antibody includes HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8. In some embodiments, the anti-PD-1 antibody includes a HCVR including an amino acid sequence of SEQ ID NO: 1. In some embodiments, the anti-PD-1 antibody includes a LCVR including an amino acid sequence of SEQ ID NO: 2. In some embodiments, the anti-PD-1 antibody includes a HCVR/LCVR amino acid sequence pair of SEQ ID NOs: 1/2. In some embodiments, the anti-PD-1 antibody includes a heavy chain and a light chain, wherein the heavy chain has an amino acid sequence of SEQ ID NO: 9. In some embodiments, the anti-PD-1 antibody includes a heavy chain and a light chain, wherein the light chain has an amino acid sequence of SEQ ID NO: 10. In some embodiments, the anti-PD-1 antibody includes a heavy chain and a light chain, wherein the heavy chain has an amino acid sequence of SEQ ID NO: 9 and the light chain has an amino acid sequence of SEQ ID NO: 10. In some embodiments, the anti-PD-1 antibody is cemiplimab.

In some embodiments, the PD-1 inhibitor is an anti-PD-1 antibody including a HCVR with at least 90% sequence identity to SEQ ID NO: 1. In some embodiments, the PD-1 inhibitor is an anti-PD-1 antibody including a LCVR with at least 90% sequence identity to SEQ ID NO: 2. In some embodiments, the PD-1 inhibitor is an anti-PD-1 antibody including a HCVR with at least 90% sequence identity to SEQ ID NO: 1, and a LCVR with at least 90% sequence identity to SEQ ID NO: 2.

In some embodiments, the method promotes tumor regression, reduces tumor cell load, reduces tumor burden, and/or prevents tumor recurrence in the patient. In some embodiments, the method leads to at least one effect selected from increase in progression-free survival, increase in overall survival, complete response, partial response, and stable disease. In some embodiments, the method leads to improved functioning and quality of life of the patient, as measured by EORTC QLQ-C30, as compared to a patient treated with chemotherapy alone. In some embodiments, the method delays the time to definitive deterioration in GHS/QOL of the patient, as measured by EORTC QLQ-C30 and QLQ-LC13, as compared to a patient treated with chemotherapy alone.

In some embodiments, the method further includes administering to the patient an additional therapeutic agent or therapy selected from one or more of: surgery, radiation, an anti-viral therapy, photodynamic therapy, a programmed death ligand 1 (PD-L1) inhibitor, a lymphocyte activation gene 3 (LAG3) inhibitor, a cytotoxic T-lymphocyte-associated protein 4 (CTLA-4) inhibitor, a glucocorticoid-induced tumor necrosis factor receptor (GITR) agonist, a T-cell immunoglobulin and mucin containing-3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand, a CD20 inhibitor, an indoleamine-2,3-dioxygenase (IDO) inhibitor, a CD28 activator, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an agonist to a co-stimulatory receptor, an antibody to a tumor-specific antigen, a vaccine, an adjuvant to increase antigen presentation, an oncolytic virus, a cytotoxin, a chemotherapeutic agent, platinum-based chemotherapy, a tyrosine kinase inhibitor, an IL-6R inhibitor, an IL-4R inhibitor, an IL-10 inhibitor, a cytokine, an antibody drug conjugate (ADC), chimeric antigen receptor T cells, an anti-inflammatory drug, and a dietary supplement.

In some embodiments, the PD-1 inhibitor is administered as one or more doses, wherein each dose is administered two weeks, three weeks, four weeks, five weeks or six weeks after the immediately preceding dose. In some embodiments, the PD-1 inhibitor is administered as two or more doses, wherein each dose is administered three weeks after the immediately preceding dose. In some embodiments, the PD-1 inhibitor is administered at a dose of 5 mg to 800 mg. In some embodiments, the PD-1 inhibitor is administered at a dose of 200 mg, 250 mg, or 350 mg. In some embodiments, the PD-1 inhibitor is administered at a dose of 1 mg/kg to 20 mg/kg of the patient's body weight. In some embodiments, the PD-1 inhibitor is administered at a dose of 1 mg/kg, 3 mg/kg or 10 mg/kg of the patient's body weight. In some embodiments, the PD-1 inhibitor is administered intravenously, or subcutaneously.

In another aspect, the disclosed technology relates to a programmed death 1 (PD-1) inhibitor for use in a method of treating or inhibiting the growth of a tumor, the method including: (a) selecting a patient with lung cancer and brain metastasis; and (b) administering to the patient a therapeutically effective amount of a programmed death-1 (PD-1) inhibitor, wherein the PD-1 inhibitor is an antibody that binds specifically to PD-1 and includes three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof.

In another aspect, the disclosed technology relates to a kit including a programmed death 1 (PD-1) inhibitor in combination with written instructions for use of a therapeutically effective amount of the PD-1 inhibitor for treating or inhibiting the growth of a tumor in a patient with lung cancer and brain metastasis.

### BRIEF DESCRIPTION OF THE DRAWINGS

FIG. 1 is a schematic of the EMPOWER-Lung 1 study design described in Example 2. Abbreviations used in FIG. 1 include: CNS, central nervous system; ECOG, Eastern Cooperative Oncology Group; HIV, human immunodeficiency virus; HRQoL, health-related quality of life; ITT, intention-to-treat; IV, intravenous; PD, progressive disease; Q3W, every 3 weeks; R, randomized; and ROW, rest of the world.

FIG. 2 is a Kaplan-Meier curve showing overall survival of patients included in the study described in Example 2.

FIG. 3 is a Kaplan-Meier curve showing progression-free survival of patients included in the study described in Example 2.

FIG. 4 is a Kaplan-Meier curve showing brain metastases progression-free survival of patients included in the study described in Example 2.

FIGS. 5A-5C are graphs showing mixed-model repeat measures (MMRM) analysis of overall least squares (LS) mean change from baseline across time points for patients included in the study described in Example 3. FIG. 5A relates to QLQ-C30 functioning scales;

FIG. 5B relates to QLQ-C30 symptom scales; and FIG. 5C relates to QLQ-LC13.

FIG. 6 is a schematic showing MMRM analysis of overall difference in treatment effects for patients included in the study described in Example 3.

FIG. 7 is a graph showing MMRM analysis of change from baseline in EORTC QLQ-C30 GHS/QOL at each cycle for patients included in the study described in Example 3.

FIG. 8 is a schematic showing hazard ratios for definitive deterioration over the study duration for patients included in the study described in Example 3.

FIG. 9 is a graph showing overall survival (OS) of patients included in the study described in Example 5. * Nominal P value.

FIG. 10 is a graph showing progression-free survival (PFS) of patients included in the study described in Example 5. * Nominal P value.

FIG. 11 is a graph showing Kaplan_meier estimated duration of response (DOR) of patients included in the study described in Example 5.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

### Methods of Treating or Inhibiting Growth of Lung Cancer

In general, lung cancer patients who have developed brain metastases constitute an underrepresented subpopulation that is often excluded from clinical trials because such patients do not tend to be responsive to anti-tumor therapy given their additional tumor burden. However, the present disclosure includes effective methods for treating or inhibiting the growth of a tumor including selecting a patient with lung cancer and brain metastases and administering to the patient in need thereof a PD-1 inhibitor, such as cemiplimab or a bioequivalent thereof. In certain embodiments, the brain metastases developed from the lung cancer. In certain embodiments, the patient has treated, stable brain metastases—e.g., the brain metastases are adequately treated and the patient has neurologically returned to baseline except for residual signs or symptoms related to the brain metastases treatment. In certain embodiments, the patient has lung cancer (e.g., NSCLC) wherein PD-L1 is expressed in ≥50% of tumor cells. In some embodiments, the lung cancer patient has no EGFR, ALK or ROS1 aberrations (i.e., the patient's tumors have tested negative for EGFR gene mutations, ALK gene translocations, and ROS1 fusions). In some embodiments, the lung cancer is locally advanced or metastatic NSCLC. In some embodiments, the lung cancer is locally advanced NSCLC. In some embodiments, the lung cancer is locally advanced NSCLC and the patient is not a candidate for surgical resection or definitive chemoradiation (e.g., definitive concurrent chemoradiation).

As used herein, “lung cancer” refers to cancer of the lung, such as non-small cell lung cancer (NSCLC) (e.g., advanced NSCLC, stage IIIB, stage IIIC, or stage IV squamous or non-squamous NSCLC, adenocarcinoma, squamous cell carcinoma, or large cell carcinoma), adenosquamous carcinoma, and sarcomatoid carcinoma. In some embodiments, the lung cancer is non-small cell lung cancer. In some embodiments, the lung cancer is squamous non-small cell lung cancer. In some embodiments, the lung cancer is non-squamous non-small cell lung cancer. In some embodiments, the lung cancer is locally advanced, recurrent or metastatic lung cancer. In some embodiments, the patient has lung cancer wherein the tumors express PD-L1 in ≥50% of tumor cells. In some embodiments, the patient has lung cancer (e.g., non-small cell lung cancer) wherein the tumors express PD-L1 in ≥50%, ≥60%, ≥70%, ≥80%, or >90% of tumor cells. In some embodiments, the patient has been previously treated with a treatment for lung cancer (e.g., an anti-tumor therapy such as chemotherapy, radiation, or a combination thereof).

As used herein, the terms “treating”, “treat”, or the like, mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for surgery, and/or to increase duration of survival of the subject. In many embodiments, the terms “tumor”, “lesion,” “tumor lesion,” “cancer,” and “malignancy” are used interchangeably and refer to one or more cancerous growths.

As used herein, the term “recurrent” refers to a frequent or repeated diagnosis of lung cancer in a patient or a frequent or repeated occurrence of individual tumors, such as primary tumors and/or new tumors that may represent recurrence of a prior tumor. In certain embodiments, administration of the PD-1 inhibitor inhibits the recurrence of a lung cancer tumor in the patient.

As used herein, the expression “a subject in need thereof” means a human or non-human mammal that exhibits one or more symptoms or indications of lung cancer, including brain metastases, and/or who has been diagnosed with lung cancer, and who needs treatment for the same. In many embodiments, the terms “subject” and “patient” are used interchangeably. The expression includes subjects with primary, established, or recurrent tumors (advanced malignancies). In specific embodiments, the expression includes human subjects that have and/or need treatment for locally advanced or metastatic lung cancer. In certain embodiments, the expression includes patients with a solid tumor that is resistant to or refractory to or is inadequately controlled by prior therapy (e.g., surgery, chemotherapy, radiation, treatment with an anti-cancer agent other than cemiplimab or a bioequivalent thereof, or a combination thereof). In certain embodiments, the expression includes subjects with lung cancer who not candidates for surgical resection or definitive chemoradiation. In certain embodiments, the expression includes subjects with lung cancer having a chronic viral infection caused by a virus, such as human immunodeficiency virus (HIV), hepatitis B virus (HBV), hepatitis C virus (HCV), human papillomavirus (HPV), cytomegalovirus (CMV), or a combination thereof, wherein the viral infection is a controlled viral infection and/or the patient is on a stable antiviral regimen.

In certain embodiments, the methods of the present disclosure are used for treating a subject with a solid tumor. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and blastomas.

In certain embodiments, the disclosed methods include administering a therapeutically effective amount of a PD-1 inhibitor (e.g., cemplimab or a bioequivalent thereof) in combination with an additional therapeutic agent or therapy. The additional therapeutic agent or therapy may be administered for increasing anti-tumor efficacy, for reducing toxic effects of one or more therapies and/or for reducing the dosage of one or more therapies. In various embodiments, the additional therapeutic agent or therapy may include one or more of: surgery, radiation, an anti-viral therapy (e.g., cidofovir), photodynamic therapy, a programmed death ligand-1 (PD-L1) inhibitor (e.g., an anti-PD-L1 antibody as disclosed in US 2015/0203580 or atezolizumab), a lymphocyte activation gene 3 (LAG3) inhibitor (e.g., an anti-LAG3 antibody), a cytotoxic T-lymphocyte-associated protein 4 (CTLA-4) inhibitor (e.g., ipilimumab), a glucocorticoid-induced tumor necrosis factor receptor (GITR) agonist (e.g., an anti-GITR antibody), a T-cell immunoglobulin and mucin containing-3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand (e.g., an antibody to CD-28, 2B4, LY108, LAIR1, ICOS, CD160 or VISTA), a CD20 inhibitor (e.g., an anti-CD20 antibody, or a bispecific CD3/CD20 antibody), an indoleamine-2,3-dioxygenase (IDO) inhibitor, a CD28 activator, a vascular endothelial growth factor (VEGF) antagonist (e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, pazopanib, or ramucirumab)), an angiopoietin-2 (Ang2) inhibitor, a transforming growth factor beta (TGFβ) inhibitor, an epidermal growth factor receptor (EGFR) inhibitor (e.g., erlotinib, cetuximab), an agonist to a co-stimulatory receptor (e.g., an agonist to CD28, 4-1BB, or OX40), an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin or a cancer vaccine), an adjuvant to increase antigen presentation (e.g., granulocyte-macrophage colony-stimulating factor), an oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), platinum-based chemotherapy (e.g., platinum-doublet chemotherapy), a tyrosine kinase inhibitor (e.g., lenvatinib, regorafenib, and cabozantinib), an IL-6R inhibitor, an IL-4R inhibitor, an IL-10 inhibitor, a cytokine such as IL-2, IL-7, IL-12, IL-21, and IL-15, an antibody drug conjugate (ADC) (e.g., anti-CD19-DM4 ADC, and anti-DS6-DM4 ADC), chimeric antigen receptor T cells (e.g., CD19-targeted T cells), an anti-inflammatory drug such as a corticosteroid, a non-steroidal anti-inflammatory drug (NSAID), and a dietary supplement such as an antioxidant.

As used herein, the term “anti-viral therapy” refers to any agent, drug or therapy used to treat, prevent, or ameliorate a viral infection in a host subject, including but not limited to: zidovudine, lamivudine, abacavir, ribavirin, lopinavir, efavirenz, cobicistat, tenofovir, rilpivirine, analgesics, corticosteroids, and combinations thereof.

In certain embodiments, administering to a subject with lung cancer and brain metastases a therapeutically effective amount of a PD-1 inhibitor (e.g., cemplimab or a bioequivalent thereof) leads to increased inhibition of tumor growth in the treated subject. In some embodiments, the disclosed methods lead to inhibition of tumor growth (e.g., tumor regression) with respect to both the lung cancer and the brain metastases. In certain embodiments, administering to a subject with lung cancer and brain metastases a therapeutically effective amount of a PD-1 inhibitor (e.g., cemplimab or a bioequivalent thereof) leads to increased tumor regression, tumor shrinkage and/or disappearance with respect to the lung cancer or with respect to both the lung cancer and the brain metastases.

In certain embodiments, the administration of a PD-1 inhibitor leads to one or more of: (i) delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years in the treated subject, as compared to an untreated subject or a subject treated with chemotherapy alone; (ii) increased disease-free survival (DFS) from date of treatment until recurrence of tumor or death, as compared to an untreated subject or a subject treated with chemotherapy alone; and (iii) improved overall response rate, complete response, or partial response, as compared to an untreated subject or a subject treated with chemotherapy alone. In certain embodiments, administering to a subject with lung cancer a therapeutically effective amount of a PD-1 inhibitor (e.g., cemplimab or a bioequivalent thereof) prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months as compared to an untreated subject or a subject treated with chemotherapy alone.

In certain embodiments, administering to a subject with lung cancer a therapeutically effective amount of a PD-1 inhibitor (e.g., cemplimab or a bioequivalent thereof) leads to increased overall survival (OS) or progression-free survival (PFS) of the subject as compared to a subject treated with chemotherapy alone. In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with chemotherapy alone. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject treated with chemotherapy alone.

PD-1 Inhibitors

The methods disclosed herein include administering a therapeutically effective amount of a PD-1 inhibitor, wherein the PD-1 inhibitor is cemiplimab (also known as REGN2810; LIBTAYO®) or a bioequivalent thereof. As used herein, the term “bioequivalent” refers to anti-PD-1 antibodies or PD-1-binding proteins or fragments thereof that are pharmaceutical equivalents or pharmaceutical alternatives whose rate and/or extent of absorption do not show a significant difference with that of cemiplimab when administered at the same molar dose under similar experimental conditions, either single dose or multiple dose. In the context of the present disclosure, the term “bioequivalent” includes antigen-binding proteins that bind to PD-1 and do not have clinically meaningful differences with cemiplimab with respect to safety, purity and/or potency.

The term “antibody,” as used herein, is intended to refer to immunoglobulin molecules included of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is included of a heavy chain variable region (“HCVR” or “VH”) and a heavy chain constant region (included of domains CH1, CH2 and CH3). Each light chain is included of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules.

As used herein, the terms “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically include at least one variable domain. The variable domain may be of any size or amino acid composition and will generally include at least one CDR which is adjacent to or in frame with one or more framework sequences. In antigen-binding fragments having a V_{H} domain associated with a V_{L} domain, the VH and VL domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}- C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may include a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the PD-1 inhibitor is an anti-PD-1 antibody (e.g., cemiplimab) including three heavy chain complementarity determining regions (HCDRs) of a heavy chain variable region (HCVR) including the amino acid sequence of SEQ ID NO: 1 and three light chain complementarity determining regions (LCDRs) of a light chain variable region (LCVR) including the amino acid sequence of SEQ ID NO: 2. According to certain embodiments, the anti-PD-1 antibody (e.g., cemiplimab) includes three HCDRs (HCDR1, HCDR2 and HCDR3) and three LCDRs (LCDR1, LCDR2 and LCDR3), wherein the HCDR1 includes the amino acid sequence of SEQ ID NO: 3; the HCDR2 includes the amino acid sequence of SEQ ID NO: 4; the HCDR3 includes the amino acid sequence of SEQ ID NO: 5; the LCDR1 includes the amino acid sequence of SEQ ID NO: 6; the LCDR2 includes the amino acid sequence of SEQ ID NO: 7; and the LCDR3 includes the amino acid sequence of SEQ ID NO: 8. In certain embodiments, the anti-PD-1 antibody (e.g., cemiplimab) includes an HCVR including SEQ ID NO: 1 and an LCVR including SEQ ID NO: 2. In certain embodiments, the anti-PD-1 antibody (e.g., cemiplimab) includes a heavy chain including the amino acid sequence of SEQ ID NO: 9 and a light chain including the amino acid sequence of SEQ ID NO: 10.

According to certain embodiments, a bioequivalent of cemiplimab is an anti-PD-1 antibody including a HCVR having at least 90%, 95%, 98% or 99% sequence identity to SEQ ID NO: 1. According to certain embodiments, a bioequivalent of cemiplimab is an anti-PD-1 antibody including a LCVR having at least 90%, 95%, 98% or 99% sequence identity to SEQ ID NO: 2. According to certain embodiments, a bioequivalent of cemiplimab is an anti-PD-1 antibody including a HCVR having at least 90%, 95%, 98% or 99% sequence identity to SEQ ID NO: 1, and a LCVR having at least 90%, 95%, 98% or 99% sequence identity to SEQ ID NO: 2. Sequence identity may be measured by methods known in the art (e.g., GAP, BESTFIT, and BLAST).

According to certain embodiments, a bioequivalent of cemiplimab is an anti-PD-1 antibody including a HCVR including an amino acid sequence of SEQ ID NO: 1 having 1-15 or more amino acid substitutions. According to certain embodiments, a bioequivalent of cemiplimab is an anti-PD-1 antibody including a LCVR including an amino acid sequence of SEQ ID NO: 2 having 1-10 or more amino acid substitutions. According to certain embodiments, a bioequivalent of cemiplimab is an anti-PD-1 antibody including a HCVR including an amino acid sequence of SEQ ID NO: 1 having 1-15 or more amino acid substitutions, and a LCVR including an amino acid sequence of SEQ ID NO: 2 having 1-10 or more amino acid substitutions.

### Pharmaceutical Compositions and Administration

The present disclosure provides therapeutic pharmaceutical compositions including the PD-1 inhibitors disclosed herein. Such pharmaceutical compositions may be formulated with suitable pharmaceutically acceptable carriers, excipients, buffers, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al., “Compendium of excipients for parenteral formulations” PDA, J Pharm Sci Technol 52:238-311 (1998).

The dose of PD-1 inhibitor (e.g., anti-PD-1 antibody) may vary depending upon the age and the size of a subject to be administered, target disease, conditions, route of administration, and the like. When a PD-1 inhibitor of the present disclosure is used for treating or inhibiting the growth of lung cancer, it may be advantageous to administer the PD-1 inhibitor at a single dose of about 0.1 to about 100 mg/kg body weight. Depending on the severity of the condition, the frequency and the duration of the treatment can be adjusted. In certain embodiments, the PD-1 inhibitor of the present disclosure can be administered as an initial dose of at least about 0.1 mg to about 800 mg, about 1 to about 600 mg, about 5 to about 500 mg, or about 10 to about 400 mg. In certain embodiments, the initial dose may be followed by administration of a second or a plurality of subsequent doses of the PD-1 inhibitor in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. The pharmaceutical composition can be also delivered in a vesicle, in particular a liposome (see, e.g., Langer (1990) Science 249:1527-1533).

The use of nanoparticles to deliver the PD-1 inhibitor of the present disclosure is also contemplated herein. Antibody-conjugated nanoparticles may be used both for therapeutic and diagnostic applications. Antibody-conjugated nanoparticles and methods of preparation and use are described in detail by Arruebo et al., 2009, “Antibody-conjugated nanoparticles for biomedical applications,” J. Nanomat., Vol. 2009, Article ID 439389, 24 pages. Nanoparticles may be developed and conjugated to antibodies contained in pharmaceutical compositions to target cells. Nanoparticles for drug delivery have also been described in, for example, U.S. Pat. No. 8,257,740 or U.S. Pat. No. 8,246,995.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, and intramuscular injections, drip infusions, etc. These injectable preparations may be prepared by methods publicly known.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. In some embodiments, the amount of the antibody contained is generally about 5 to about 600 mg per dosage form in a unit dose, such as about 5 to about 350 mg, or about 10 to about 300 mg.

In certain embodiments, the present disclosure provides a pharmaceutical composition or formulation including a therapeutic amount of a PD-1 inhibitor (e.g., cemplimab or a bioequivalent thereof) and a pharmaceutically acceptable carrier. Non-limiting examples of pharmaceutical compositions including an anti-PD-1 antibody provided herein that can be used in the context of the present disclosure are disclosed in US 2019/0040137.

The present disclosure also provides kits comprising a PD-1 inhibitor (e.g., cemplimab or a bioequivalent thereof) for therapeutic uses as described herein. Kits typically include a label indicating the intended use of the contents of the kit and instructions for use. As used herein, the term “label” includes any writing, or recorded material supplied on, in or with the kit, or which otherwise accompanies the kit. Accordingly, this disclosure provides a kit for treating a patient afflicted with lung cancer and brain metastasis, the kit comprising: (a) a therapeutically effective dosage of a PD-1 inhibitor (e.g., cemplimab or a bioequivalent thereof); and (b) instructions for using the PD-1 inhibitor in any of the methods disclosed herein.

### Administration Regimens

In certain embodiments, the methods disclosed herein include administering to the tumor of a subject in need thereof a therapeutically effective amount of a PD-1 inhibitor (e.g., cemiplimab or a bioequivalent thereof) in multiple doses, e.g., as part of a specific therapeutic dosing regimen. For example, the therapeutic dosing regimen may include administering one or more doses of a PD-1 inhibitor to the subject at a frequency of about once a day, once every two days, once every three days, once every four days, once every five days, once every six days, once a week, once every two weeks, once every three weeks, once every four weeks, once every five weeks, once every six weeks, once every eight weeks, once every twelve weeks, once a month, once every two months, once every three months, once every four months, twice a day, twice every two days, twice every three days, twice every four days, twice every five days, twice every six days, twice a week, twice every two weeks, twice every three weeks, twice every four weeks, twice every five weeks, twice every six weeks, twice every eight weeks, twice every twelve weeks, twice a month, twice every two months, twice every three months, twice every four months, three times a day, three times every two days, three times every three days, three times every four days, three times every five days, three times every six days, three times a week, three times every two weeks, three times every three weeks, three times every four weeks, three times every five weeks, three times every six weeks, three times every eight weeks, three times every twelve weeks, three times a month, three times every two months, three times every three months, three times every four months or less frequently or as needed so long as a therapeutic response is achieved.

In certain embodiments, the one or more doses are administered in at least one treatment cycle—e.g., 1, 2, 3, 4, 5, 6, 7, 8, 9, or 10 treatment cycles. In certain embodiments, each dose of the PD-1 inhibitor includes 0.1, 1, 0.3, 3, 4, 5, 6, 7, 8, 9 or 10 mg/kg of the patient's body weight. In certain embodiments, each dose includes about 5 to 800 mg of the PD-1 inhibitor, for example about 5, 10, 15, 20, 25, 40, 45, 50, 60, 70, 80, 90, 100, 150, 200, 250, 300, 350, 400, 450, 500, 550, 600, 650, 700, 750 mg or more of the PD-1 inhibitor.

### Dosage

The amount of PD-1 inhibitor (e.g., cemiplimab or a bioequivalent thereof) administered to a subject according to the methods disclosed herein is, generally, a therapeutically effective amount. As used herein, the term “therapeutically effective amount” means an amount of a PD-1 inhibitor administered to a patient for treating lung cancer that results in one or more of: (a) inhibition of tumor growth, or an increase in tumor necrosis, tumor shrinkage and/or tumor disappearance; (b) a reduction in the severity or duration of a symptom or an indication of lung cancer—e.g., a tumor lesion; (c) delay in tumor growth and development; (d) inhibition of tumor metastasis; (e) prevention of recurrence of tumor growth; (f) increase in survival of a subject with lung cancer; and/or (g) delay of surgery, each as compared to an untreated subject or a subject treated with chemotherapy alone.

In certain embodiments, a therapeutically effective amount of the PD-1 inhibitor (e.g., cemplimab or a bioequivalent thereof) can be from about 0.05 mg to about 800 mg, from about 1 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the PD-1 inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg.

The amount of a PD-1 inhibitor (e.g., cemplimab or a bioequivalent thereof) contained within an individual dose may be expressed in terms of milligrams of antibody per kilogram of subject body weight (i.e., mg/kg). In certain embodiments, the PD-1 inhibitor used in the methods disclosed herein may be administered to a subject at a dose of about 0.0001 to about 100 mg/kg of subject body weight. In certain embodiments, an anti-PD-1 antibody may be administered at dose of about 0.1 mg/kg to about 20 mg/kg of a patient's body weight. In certain embodiments, the methods of the present disclosure include administration of a PD-1 inhibitor (e.g., an anti-PD-1 antibody) at a dose of about 1 mg/kg to 3 mg/kg, 1 mg/kg to 5 mg/kg, 1 mg/kg to 10 mg/kg, 1 mg/kg, 3 mg/kg, 5 mg/kg, or 10 mg/kg of a patient's body weight.

In certain embodiments, an individual dose amount of a PD-1 inhibitor (e.g., cemplimab or a bioequivalent thereof) administered to a patient may be less than a therapeutically effective amount, i.e., a subtherapeutic dose. For example, if the therapeutically effective amount of a PD-1 inhibitor includes 3 mg/kg, a subtherapeutic dose includes an amount less than 3 mg/kg, e.g., 2 mg/kg, 1.5 mg/kg, 1 mg/kg, 0.5 mg/kg or 0.3 mg/kg. As defined herein, a “subtherapeutic dose” refers to an amount of the PD-1 inhibitor that does not lead to a therapeutic effect by itself. However, in certain embodiments, multiple subtherapeutic doses of a PD-1 inhibitor are administered to collectively achieve a therapeutic effect in the subject.

In certain embodiments, each dose includes 0.1-10 mg/kg (e.g., 0.3 mg/kg, 1 mg/kg, 3 mg/kg, or 10 mg/kg) of PD-1 inhibitor (e.g., cemplimab or a bioequivalent thereof) based on the subject's body weight. In certain other embodiments, each dose includes 5 to 600 mg of the PD-1 inhibitor, e.g., 5 mg, 10 mg, 15 mg, 20 mg, 25 mg, 30 mg, 40 mg, 45 mg, 50 mg, 100 mg, 150 mg, 200 mg, 250 mg, 300 mg, 400 mg, or 500 mg.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Clinical Trial of Cemiplimab for the Treatment of Advanced or Metastatic Non-Small Cell Lung Cancer (NSCLC)

This example describes a randomized, global, open-label, phase 3 study of cemiplimab monotherapy versus standard-of-care, platinum-based, doublet chemotherapies in patients with advanced or metastatic, squamous or non-squamous NSCLC whose tumors express PD-L1 in >50% of tumor cells and who have received no prior systemic treatment for their advanced disease. The overall goal of the study is to assess safety and efficacy of cemiplimab as first-line treatment in patients with advanced or metastatic NSCLC whose tumors highly express PD-L1. The main objectives of the study are to determine if cemiplimab improves OS and/or PFS over standard-of-care platinum doublet chemotherapy in this patient population. Additional objectives include further characterization of tumor responses, patient-reported outcomes, safety, and pharmacokinetics (PK).

Objectives: A primary objective of the study is to compare the OS of cemiplimab versus standard-of-care platinum-based chemotherapies in the first-line treatment of patients with advanced or metastatic NSCLC whose tumors express PD-L1 in ≥50% of tumor cells. Overall survival has been recognized as the gold standard for demonstrating benefit of antineoplastic therapies in randomized clinical trials. Another primary objective of the study is to compare the PFS of cemiplimab versus standard-of-care platinum-based chemotherapies in the first-line treatment of patients with advanced or metastatic NSCLC whose tumors express PD-L1 in >50% of tumor cells. Progression-free survival, the time to tumor progression (based on Response Evaluation Criteria in Solid Tumors version 1.1 [RECIST 1.1] criteria [Eisenhauer) 2009]) or death, was chosen as a primary endpoint because PFS is recognized as a marker of clinical benefit. Based on data released for another PD-1 antibody, PFS would be expected to be prolonged in patients with NSCLC whose tumors express PD-L1 in >50% of tumor cells. Disease progression will be determined based on the RECIST 1.1 criteria (Eisenhauer 2009). The first radiographic tumor assessment will occur after 9 weeks of study treatment and every 9 weeks thereafter. A secondary objective of the study includes comparing the ORR of cemiplimab versus platinum-based chemotherapies.

Efficacy responses to be assessed include assessment of ORR and DOR to treatment. From a patient perspective, preservation of QOL is important; therefore, QOL is assessed through the use of two validated questionnaires, EORTC QLQ-C30 and EORTC QLQ-LC13 (Bergman 1994, Bjordal 2000).

It is expected that the study will show that cemiplimab prolongs OS as compared with standard of care platinum-based chemotherapies in the first line treatment of patients with advanced or metastatic NSCLC whose tumors PD-L1 in ≥50% of tumor cells; and that cemiplimab prolongs PFS as compared with standard of care platinum-based chemotherapies in the first line treatment of patients with advanced or metastatic NSCLC whose tumors PD-L1 in ≥50% of tumor cells.

Rationale: The population selected for inclusion in this study represents a large proportion of the patients with newly diagnosed advanced lung cancer for whom the first-line, standard-of-care treatment option is chemotherapy. Despite available therapies, the disease often progresses. Subsequent treatment options have been limited, and the 5-year survival is low with few apparent cures. NSCLC tumors express PD-L1, and preliminary data suggest that use of PD-1/PD-L1 inhibitors in first-line treatment either as monotherapy or in combination with chemotherapy may benefit patients whose tumors express high levels of PD-L1 (compared with those that either do not express PD-L1 or express low levels of PD-L1); hence, the focus on including only patients whose tumors express PD-L1 in ≥50% of tumor cells. The study is open-label because chemotherapy is administered for a limited timeframe compared with cemiplimab, which is intended to be given for up to 2 years. Imposing a double-blind design would require patients in the chemotherapy treatment group to receive placebo until the time of disease progression, and this was not viewed as acceptable given the nature of the disease. Patients in the chemotherapy treatment group will be given the option to crossover to cemiplimab monotherapy at the time of disease progression given that use of PD-1 inhibitor is now the standard-of-care for second-line treatment of NSCLC (Ettinger 2016).

Regarding the rationale for standard-of-care chemotherapy as a comparator, platinum-based doublet chemotherapy is currently recommended as first-line treatment for advanced or metastatic NSCLC (Reck 2014, Ettinger 2016), and therefore serves as the active comparator in this study. There is no single “best” platinum-based doublet standard chemotherapy for squamous or non-squamous NSCLC. Randomized studies that have compared various regimens have not shown any difference in survival (Fossella 2003, Scagliotti 2002, Schiller 2002). Pemetrexed-based doublets are restricted to non-squamous NSCLC (ALIMTA® US PI, ALIMTA European Union Summary of Product Characteristics).

Both cisplatin and carboplatin are used, although carboplatin may be associated with fewer side effects. Randomized, controlled studies of patients with stage IV disease and good performance status have shown that cisplatin-based chemotherapy improves survival and palliates disease-related symptoms (Weick 1991). Administration of 4 cycles of chemotherapy is standard, but up to 6 cycles may be given in non-progressing patients that are tolerating treatment. For this study, patients are administered chemotherapy for at least 4 cycles and up to 6 cycles, depending on patient tolerability and disease assessment.

Primary Endpoints: The primary endpoints are OS and PFS (per RECIST 1.1, Eisenhauer 2009). Overall survival is defined as the time from randomization to the date of death. A patient who has not died is censored at the last known date of contact. Progression-free survival is defined as the time from randomization to the date of the first documented tumor progression or death due to any cause. Patients are censored according to the following rules: (1) patients who do not have a documented tumor progression or death are censored on the date of their last evaluable tumor assessment; (2) patients who do not have a documented tumor progression or death before initiation of new anti-tumor therapy are censored on the date of their last evaluable tumor assessment prior to or on the date of new anti-tumor therapy; (3) patients who withdraw consent before taking any study treatment, and as a consequence have no post-baseline tumor assessment, are censored at the date of randomization; (4) patients who do not have any evaluable tumor assessments after randomization and do not die are censored on the date of randomization.

Secondary Endpoints: A key secondary endpoint in the study is ORR. Objective response rate is defined as the number of patients with a best overall response (BOR) of confirmed CR or PR divided by the number of patients in the efficacy analysis set. Best overall response is defined as the best overall response (per RECIST 1.1) between the date of randomization and the date of the first objectively documented progression or the date of subsequent anti-cancer therapy, whichever comes first. Other secondary endpoints include DOR and QOL. Duration of response is defined as the time between the date of first response (CR or PR) to the date of the first documented tumor progression (per RECIST 1.1) or death due to any cause. Quality of life is measured by EORTC QLQ-C30 and EORTC QLQ-LC13. Further secondary endpoints also include the safety and tolerability of cemiplimab. To evaluate the safety and tolerability of cemiplimab versus platinum-based chemotherapies, safety and tolerability are measured by the incidence of AEs, serious adverse events (SAEs), deaths, and laboratory abnormalities to assess immunogenicity (ADA/Nabs) to cemiplimab and any relationship with drug concentrations, efficacy and safety.

Study Design: This is a randomized, multicenter, open-label, pivotal phase 3 study of cemiplimab monotherapy versus platinum-based doublet chemotherapy in patients with stage IIIB or IIIC, or stage IV squamous or non-squamous NSCLC whose tumors express PD-L1 in >50% of tumor cells and who have received no prior systemic treatment for their advanced disease. The study includes the following 3 periods: screening, treatment, and follow-up.

Screening: Patients undergo a screening evaluation to determine their eligibility within 28 days prior to randomization. PD-L1 expression in tumor tissue (archival or recently obtained biopsy collected) is assessed using a validated PD-L1 IHC assay that reports 50% positivity. Patients whose tumors express PD-L1 in ≥50% of tumor cells continue in screening. Patients whose tumors express PD-L1 in <50% of tumor cells are excluded from the study. Tumor tissue are also tested for mutations in epidermal growth factor receptor (EGFR) and anaplastic lymphoma kinase (ALK) and for human homolog of the transforming gene v-ros of the avian sarcoma virus UR2 (ROS1) fusions. Baseline radiographic tumor assessments are also performed within 28 days prior to randomization.

Treatment: Eligible patients are randomized to one of the following 2 treatment groups: (i) Cemiplimab 350 mg monotherapy; or (ii) Standard-of-care chemotherapy, as summarized in Table 1.

TABLE 1
Schedule of Events: Treatment Period/On-Study Assessments and Procedures

| | Year 1 | | | Year 2 | | |
|---|---|---|---|---|---|---|
| | | Day 1, | Every 3 | Day 1, | | |
| | | Each Cycle | Cycles | Each Cycle | Every 3 | |
| Treatment | | (Every | (9 Weeks | (Every | Cycles | |
| (one of the | Day 1, | 21 Days ± | [63 Days] ± | 21 Days ± | (9 Weeks ± | |
| following) | Cycle 1 | 3 Days) | 5 Days) | 3 Days) | 5 Days) | Comments |
| Cemiplimab | X | X | | X | | Administer Q3W for up to |
| 350 mg | | | | | | 108 weeks or until disease |
| | | | | | | progression. |
| Paclitaxel | X | X | | | | Administer 4 to 6 cycles. |
| Carboplatin | X | X | | | | Administer 4 to 6 cycles. |
| Cisplatin | X | X | | | | Administer 4 to 6 cycles. |
| Gemcitabine | X | X | | | | Administer on days 1 and 8 of |
| | | | | | | each 21-day cycle. |
| Pemetrexed | X | X | | | | Optionally administer on day |
| | | | | | | 1 every 21 days until disease |
| | | | | | | progression. |

Randomization is stratified by histology (non-squamous versus squamous) and geographic region (Europe, Asia, or Rest of World [ROW]). Treatment begins within 3 days of randomization. Details of the treatment regimens are provided below. Patients with NSCLC randomized to chemotherapy may receive one of the following regimens: (i) Paclitaxel+cisplatin or carboplatin; (ii) Gemcitabine+cisplatin or carboplatin; or (iii) Pemetrexed+cisplatin or carboplatin followed by optional pemetrexed maintenance (it is strongly recommended that patients with squamous NSCLC do not receive pemetrexed-containing regimens). For the purposes of this study, a treatment cycle is defined as 21 days or 3 weeks.

Based on the demonstrated improvement in OS in patients receiving cemiplimab, all patients randomized to receive platinum-based chemotherapy are permitted to cross over to receive treatment with cemiplimab 350 mg Q3W for up to 108 additional weeks (36 cycles) prior to disease progression. Patients eligible to cross over to cemiplimab include the following: (i) patients who are actively being treated with chemotherapy; (ii) patients who have completed chemotherapy and are in follow-up, but whose disease has not yet progressed; and (iii) patients who have discontinued chemotherapy and have progressed, but have not yet crossed over to cemiplimab for any reason. All other patients continue study treatment until either their disease progresses or they complete 108 weeks of treatment with cemiplimab.

Treatment Period—Cemiplimab Patients: Patients assigned to the cemiplimab treatment group receive cemiplimab 350 mg as an IV infusion on day 1 of every treatment cycle for up to 108 weeks or until RECIST 1.1-defined progressive disease, unacceptable toxicity, death, or withdrawal of consent. Radiographic tumor assessments are obtained in all patients every 3 cycles beginning at week 9 (day 63±5 days), until disease progression, loss to follow-up, withdrawal of consent, death, or initiation of another anti-cancer treatment. RECIST 1.1-defined progressive disease is required to declare progression of the purposes of PFS, ORR, and DOR endpoints, and for the purpose of receiving extended treatment of chemotherapy in addition to cemiplimab. Safety is assessed through the occurrence of TEAEs, vital sign evaluation, physical examination, and laboratory analyses. Blood samples are collected to measure serum concentrations of cemiplimab and ADA and NAb titers. To assess disease-related symptoms, patients are asked to complete QOL questionnaires at specified time points.

Treatment Period—Chemotherapy Patients: Patients assigned to chemotherapy receive one of the options of platinum-doublet chemotherapy treatment for 4 to 6 cycles or until RECIST 1.1-defined progressive disease, unacceptable toxicity, death, or withdrawal of consent. In some instances, patients with non-squamous NSCLC treated with pemetrexed and cisplatin or carboplatin may receive pemetrexed maintenance therapy until disease progression. Radiographic tumor assessments are obtained in all patients beginning at week 9 (day 63±5 days) every 3 cycles until disease progression, loss to follow-up, withdrawal of consent, death, or initiation of another anti-cancer treatment. Disease progression is defined using RECIST 1.1 criteria. Disease progression and BOR are used for endpoint assessments. Safety is assessed through the occurrence of TEAEs, vital sign evaluation, physical examination, and laboratory analyses. To assess disease-related symptoms, patients are asked to complete QOL questionnaires at specified time points. All patients are permitted to cross over to receive cemiplimab 350 mg Q3W for up to 108 additional weeks prior to disease progression.

The best overall response (BOR) is the best response recorded from the start of the treatment until the end of treatment taking into account any requirement for confirmation. The patient's best response assignment will depend on the findings of both target and non-target disease and will also take into consideration the appearance of new lesions, as summarized in Table 2.

TABLE 2
Response According to Revised Response Evaluation
Criteria in Solid Tumors (Version 1.1) in Patients
with Target (and Non-Target) Lesions

| Target | | | Overall |
|---|---|---|---|
| Lesions | Non-target Lesions | New Lesions | Response |
| CR | CR | No | CR |
| CR | Non-CR/non-PD | No | PR |
| CR | Not evaluated | No | PR |
| PR | Non-CR/non-PD/not evaluated | No | PR |
| SD | Non-CR/non-PD/not evaluated | No | SD |
| PD | Any | Yes or no | PD |
| Any | PD* | Yes or no | PD |
| Any | Any | Yes | PD |

CR = complete response;
PD = progressive disease;
PR = partial response;
SD = stable disease

Follow-Up: Patients have follow-up visits every 6 weeks for approximately 6 months and then at 9 months and 12 months after the last dose of treatment. The study ends when the survival analysis is complete. The last patient last visit is 48 months from the enrollment of the last patient. The duration of the study for each patient is approximately 48 months.

Follow-Up Period—Cemiplimab Patients: Patients in the cemiplimab treatment group enter the follow-up period after completion of the 108-week treatment period or when the decision is made to discontinue cemiplimab therapy. Patients who discontinued cemiplimab treatment early due to CR or PR, who then have disease progression while in follow-up may optionally begin retreatment with cemiplimab 350 mg Q3W for up to 108 weeks. Patients assigned to the cemiplimab treatment group have blood samples taken for PK and ADA testing.

Follow-up Period—Chemotherapy Patients: Patients in the chemotherapy treatment group enter follow-up after completion of 4 to 6 cycles, at the time of initial RECIST 1.1-defined progressive disease while on therapy, or early termination. All patients are permitted to cross over to receive cemiplimab 350 mg Q3W for up to 108 additional weeks prior to disease progression. Patients that opt to be treated with anti-cancer treatments including cemiplimab, other than chemotherapy, are expected to complete all follow-up assessments.

Follow-up Period—Chemotherapy Crossover Patients: Patients who experience RECIST 1.1-defined progressive disease while on chemotherapy or after completion of chemotherapy are offered the option at the time of progression to receive cemiplimab 350 mg Q3W for up to 108 weeks, provided they meet the criteria for cemiplimab therapy. Patients who crossover to receive cemiplimab 350 mg Q3W for up to 108 weeks enter follow-up after completion of up to 108 weeks of treatment, at the time of progression while on therapy, or early termination. All patients are permitted to crossover to receive cemiplimab 350 mg Q3W for up to 108 additional weeks (36 cycles) prior to disease progression.

Extended Treatment—Cemiplimab Continuation plus Chemotherapy Patients: Patients originally assigned to receive cemiplimab, who experience RECIST 1.1-defined progressive disease on therapy may continue treatment with cemiplimab 350 mg Q3W for up to 108 additional weeks, along with the addition of histology-specific chemotherapy for 4 cycles, provided they meet specific criteria and the patient has not completed the 108-week treatment period. Alternatively, these patients may opt to initiate a new anti-cancer treatment. Histology-specific chemotherapy is defined as paclitaxel plus cisplatin or carboplatin for squamous cell NSCLC and pemetrexed plus cisplatin or carboplatin followed by optional pemetrexed maintenance for non-squamous NSCLC. Patients who are treated with cemiplimab plus histology-specific chemotherapy beyond the initial determination of progressive disease enter follow-up after completion of up to 108 additional weeks of treatment, at the time of further progression while on therapy, or early termination.

Study Patient Population: Patients included in this study are men and women ≥18 years of age, diagnosed with stage IIIB or IIIC or stage IV squamous or non-squamous NSCLC, who are not candidates for definitive chemotherapy and radiation, whose tumors express PD-L1 in >50% of tumor cells (using the PD-L1 IHC 22C3 pharmDx assay) and who have received no prior systemic treatment for their advanced disease. The study population is limited to previous and current smokers. Patients with actionable mutations are excluded.

Inclusion Criteria: A patient must meet the following criteria to be eligible for inclusion in the study: (1) Men and women ≥18 years of age; (2) Patients with histologically or cytologically documented squamous or non-squamous NSCLC with stage IIIB or stage IIIC disease who are not candidates for treatment with definitive concurrent chemoradiation or patients with untreated stage IV disease who received no prior systemic treatment for recurrent or metastatic NSCLC. Patients who received adjuvant or neoadjuvant platinum-doublet chemotherapy (after surgery and/or radiation therapy) and developed recurrent or metastatic disease more than 6 months after completing therapy are eligible; (3) Archival or newly obtained formalin-fixed tumor tissue from a metastatic/recurrent site, which has not previously been irradiated. Tissue may be obtained from the primary site if it is still in place and the other metastatic sites are either not accessible (i.e., brain), cannot be used (i.e., bone), or the biopsy would put the patient at undue risk. If an archival biopsy is used, it must be less than 5 months old; (4) Tumor cells expressing PD-L1 in ≥50% of tumor cells by IHC performed by the central laboratory; (5) At least 1 radiographically measurable lesion by computed tomography (CT) or magnetic resonance imaging (MRI) per RECIST 1.1 criteria. Target lesions may be located in a previously irradiated field if there is documented (radiographic) disease progression in that site; (6) ECOG performance status of ≤1; (7) Anticipated life expectancy of at least 3 months; (8) Adequate organ and bone marrow function defined as follows: (a) Hemoglobin ≥9.0 g/dL; (b) Absolute neutrophil count ≥1.5×10⁹/L; (c) Platelet count ≥100,000/mm³; (d) Glomerular filtration rate (GFR) >30 mL/min/1.73 m²; (e) Total bilirubin≤1.5×upper limit of normal (ULN) (if liver metastases≤3 ×ULN), with the exception of patients diagnosed with clinically confirmed Gilbert's syndrome; (f) Aspartate aminotransferase (AST) and alanine aminotransferase (ALT) ≤3×ULN or ≤5×ULN, if liver metastases; (g) Alkaline phosphatase ≤2.5×ULN (or ≤5.0×ULN, if liver or bone metastases); (h) Not meeting criteria for Hy's law (ALT >3×ULN and bilirubin >2×ULN); (9) Willing and able to comply with clinic visits and study-related procedures; (10); Provide signed informed consent; and (11) Able to understand and complete study-related questionnaires.

Exclusion Criteria: A patient who meets any of the following criteria will be excluded from the study: (1) Patients that have never smoked, defined as smoking ≤100 cigarettes in a lifetime; (2) Active or untreated brain metastases or spinal cord compression. Patients are eligible if central nervous system (CNS) metastases are adequately treated and patients have neurologically returned to baseline (except for residual signs or symptoms related to the CNS treatment) for at least 2 weeks prior to randomization. Patients must be off (immunosuppressive doses of) corticosteroid therapy; (3) Patients with tumors tested positive for EGFR gene mutations, ALK gene translocations, or ROS1 fusions. All patients should have tumor evaluations for EGFR mutations, ALK rearrangement, and ROS1 fusions confirmed by a central laboratory; (4) Encephalitis, meningitis, or uncontrolled seizures in the year prior to randomization; (5) History of interstitial lung disease (e.g., idiopathic pulmonary fibrosis, organizing pneumonia) or active, noninfectious pneumonitis that required immune-suppressive doses of glucocorticoids to assist with management. A history of radiation pneumonitis in the radiation field is permitted as long as pneumonitis resolved ≥6 months prior to randomization; (6) Patients with active, known, or suspected autoimmune disease that has required systemic therapy in the past 2 years. Patients with vitiligo, type I diabetes mellitus, and hypothyroidism (including hypothyroidism due to autoimmune thyroiditis) only requiring hormone replacement are permitted to be randomized; (7) Patients with a condition requiring corticosteroid therapy (>10 mg prednisone/day or equivalent) within 14 days of randomization. Physiologic replacement doses are allowed even if they are >10 mg of prednisone/day or equivalent, as long as they are not being administered for immunosuppressive intent. Inhaled or topical steroids are permitted, provided that they are not for treatment of an autoimmune disorder; (8) Another malignancy that is progressing or requires treatment, with the exception of nonmelanomatous skin cancer that has undergone potentially curative therapy, or in situ cervical carcinoma or any other tumor that has been treated, and the patient is deemed to be in complete remission for at least 2 years prior to randomization, and no additional therapy is required during the study period; (9) Uncontrolled infection with hepatitis B or hepatitis C or human immunodeficiency virus; or diagnosis of immunodeficiency. (a) Patients with hepatitis B (HepBsAg+) who have controlled infection (serum hepatitis B virus DNA PCR that is below the limit of detection AND receiving anti-viral therapy for hepatitis B) are permitted. (b) Patients who are hepatitis C virus antibody positive (HCV Ab+) who have controlled infection (undetectable HCV RNA by PCR either spontaneously or in response to a successful prior course of anti-HCV therapy) are permitted. (c) Patients with HIV who have controlled infection (undetectable viral load and CD4 count above 350 either spontaneously or on a stable antiviral regimen) are permitted; (10) Active infection requiring systemic therapy within 14 days prior to randomization; (11) Prior therapy with anti-PD-1 or anti-PD-L1. Prior exposure to other immunomodulatory or vaccine therapy such as anti-cytotoxic T-lymphocyte-associated antigen 4 (CTLA-4) antibodies is permitted, but the last dose of such an antibody should have been at least 3 months prior to the first dose of study drug; (12) Treatment-related immune-mediated AEs from immune-modulatory agents (including but not limited to anti-PD1/PD-L1 mAbs, anti-CTLA4 mAbs, and phosphoinositol 3-kinase [PI 3-K]-δ inhibitors) that have not resolved to baseline at least 3 months prior to initiation of treatment with study therapy. Patients are excluded from treatment with cemiplimab if they experienced immune-mediated AEs related to prior treatment with a blocker of the PD-1/PD-L1 pathway that were grade 3 or 4 in severity and/or required discontinuation of the agent, regardless of time of occurrence; (13) Receipt of an investigational drug or device within 30 days of screening or within 5 half-lives of the investigational drug (whichever is longer); (14) Receipt of a live vaccine within 30 days of planned start of study medication; (15) Major surgery or significant traumatic injury within 4 weeks prior to first dose; (16) Documented allergic or acute hypersensitivity reaction attributed to antibody treatments; (17) Known psychiatric or substance abuse disorder that would interfere with participation with the requirements of the study, including current use of any illicit drugs; (18) Pregnant or breastfeeding women; (19) Women of childbearing potential or men who are unwilling to practice highly effective contraception prior to the initial dose/start of the first treatment, during the study, and for at least 6 months after the last dose. Highly effective contraceptive measures include: stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; intrauterine device (IUD); intrauterine hormone-releasing system (IUS); bilateral tubal ligation; vasectomized partner (provided that partner is the sole sexual partner of the WOCBP patient and that the vasectomized partner has received medical assessment of the surgical success); and/or sexual abstinence. Women of child bearing potential is defined as women who are fertile, following menarche and until becoming post-menopausal unless permanently sterile. Permanent sterilisation methods include hysterectomy, bilateral salpingectomy and bilateral oophorectomy. A postmenopausal state is defined as no menses for 12 months without an alternative medical cause. A high follicle stimulating hormone (FSH) level in the postmenopausal range may be used to confirm a post-menopausal state in women not using hormonal contraception or hormonal replacement therapy. However in the absence of 12 months of amenorrhea, a single FSH measurement is insufficient. Pregnancy testing and contraception are not required for women with documented hysterectomy or tubal ligation; (20) Patients who are committed to an institution by virtue of an order issued either by the judicial or the administrative authorities; (21) Prior treatment with idelalisib; (22) Member of the clinical site study team and/or his/her immediate family, unless prior approval granted; (23) Recipients of organ transplants; (24) Active or latent tuberculosis. Latency should be ruled out by purified protein derivative (PPD)/QuantiFERON testing in high risk individuals.

Study Treatments: Cemiplimab (REGN2810) is supplied as a liquid in sterile, single-use vials. Each vial contains cemiplimab at a concentration of 50 mg/mL. Cemiplimab is administered in an outpatient setting as a 30-minute (+10 minutes) IV infusion. Study treatment (cemiplimab or chemotherapy) is administered to patients randomized to receive one of the following treatment regimens: Cemiplimab at 350 mg as an IV infusion or Platinum-based doublet chemotherapy (with or without maintenance therapy). Cemiplimab is administered Q3W (every 3 weeks) for up to 108 weeks or until disease progression, unacceptable toxicity, death, or withdrawal of consent. As summarized in Table 3, chemotherapy will be administered for 4 to 6 cycles, or until disease progression, unacceptable toxicity, death, or withdrawal of consent, and the chemotherapy option will be one selected regimen.

TABLE 3
Platinum-Based Doublet Chemotherapy Regimens

| Option | Chemotherapy Regimen | Dosing Frequency | Maintenance Therapy |
|---|---|---|---|
| 1 | Pemetrexed 500 mg/m² IV | Day 1 every 21 days for 4 to | Optional pemetrexed |
| | plus cisplatin 75 mg/m²IV | 6 cycles | 500 mg/m² IV day 1 |
| | | | every 21 days |
| 2 | Pemetrexed 500 mg/m² IV | Day 1 every 21 days for 4 to | Optional pemetrexed |
| | plus carboplatin AUC of | 6 cycles | 500 mg/m² IV day 1 |
| | 5 or 6 mg/mL/minute IV | | every 21 days |
| 3 | Paclitaxel 200 mg/m² IV plus | Day 1 every 21 days for 4 to | No maintenance |
| | cisplatin 75 mg/m² IV | 6 cycles | |
| 4 | Paclitaxel 200 mg/m² IV plus | Day 1 every 21 days for 4 to | No maintenance |
| | carboplatin AUC of | 6 cycles | |
| | 5 or 6 mg/mL/minute IV | | |
| 5 | Gemcitabine 1250 mg/m² IV | Day 1 and day 8 (gemcitabine | No maintenance |
| | plus cisplatin 100 mg/m² IV | only) every 21 days for 4 to | |
| | | 6 cycles | |
| 6 | Gemcitabine 1250 mg/m² IV | Day 1 and day 8 (gemcitabine | No maintenance |
| | plus carboplatin AUC of | only) every 21 days for 4 to | |
| | 5 or 6 mg/mL/minute IV | 6 cycles | |

AUC = area under the curve;
IV = intravenous;
N/A = not applicable

Concomitant Medications and Procedures: Any procedure performed, or treatment administered from the time of informed consent until 90 days after the last study treatment (cemiplimab or chemotherapy) is considered concomitant medication. This includes medications that were started before the study and are ongoing during the study, as well as any therapies started in the follow-up period to treat a study-drug-related AE.

Prohibited Medications: While participating in this study, a patient may not receive any investigational agent for treatment of a tumor other than cemiplimab as monotherapy or the study's specified chemotherapy regimens. Treatment with bevacizumab or necitumumab is not one of the protocol-defined treatment options. Any other medication that is considered necessary for the patient's welfare and is not expected to interfere with the evaluation of the cemiplimab may be given at the discretion of the investigator.

Permitted Medications and Procedures: It is recommended that patients do not receive concomitant systemic corticosteroids such as hydrocortisone, prednisone, prednisolone, or dexamethasone at any time throughout the study, except in the case of a life-threatening emergency and/or to treat an irAE. Physiologic replacement doses of systemic corticosteroids are permitted, even if >10 mg/day prednisone equivalents. A brief course of corticosteroids for prophylaxis (e.g., contrast dye allergy) or for treatment of non-autoimmune conditions (e.g., delayed-type hypersensitivity reaction caused by contact allergen) is permitted. Treatments for bone metastases (bisphosphonates, denosumab) are permitted. Pemetrexed maintenance therapy is permitted for non-squamous NSCLC. Radiation therapy with palliative intent is permitted.

Study Procedures-Screening: For collection of tumor tissue for PD-L1 assessment using the PD-L1 IHC 22C3 pharmDx assay, a formalin-fixed, paraffin-embedded tissue block or unstained slides of tumor sample (archival or recent) is provided. Biopsies should be of sufficient size to ensure an adequate amount of tissue for analysis (excisional or incisional). Tumor tissue is also tested for mutations in EGFR and ALK and for ROS1 fusions at a central laboratory. Randomization is based on local confirmation of histology. Screening procedures also include baseline radiographic tumor assessment of the chest, abdomen, pelvis, and all other known or suspected sites of disease by CT or MRI; chest X-ray PA/Lateral; serum pregnancy test in women of childbearing potential within 72 hours prior to administration of the first study treatment administration; and TB testing for high risk individuals using purified protein derivative (PPD)/QuantiFERON testing.

Study Procedures-Efficacy: For radiographic tumor assessments, high-resolution CT with contrast and contrast-enhanced MRI are the preferred imaging modalities for assessing radiographic tumor response. In patients in whom contrast is strictly contraindicated, non-contrast scans will suffice. The chest, abdomen, and pelvis are imaged along with any other known or suspected sites of disease. If more than one imaging modality is used at screening, the most accurate imaging modality according to RECIST 1.1 should be used when recording data. The same imaging modality used at screening should be used for all subsequent assessments. Radiographic tumor assessments are obtained in all patients at every 3 cycles, at week 9 (day 63±5 days) and every 9 weeks thereafter, until disease progression, loss to follow-up, withdrawal of consent, death, or initiation of another anti-cancer treatment. Tumor measurements are made in accordance with RECIST 1.1 criteria (Eisenhauer 2009). For Quality of Life Questionnaires, patient-reported outcomes are measured using the following validated patient self-administered questionnaires: EORTC QLQ-C30 and EORTC QLQ-LC13 (Bergman 1994, Bjordal 2000). Patients are asked to complete these questionnaires prior to any study procedures being performed at a given study visit (during the on-study/treatment and follow-up periods).

### Example 2: Results of Clinical Trial of Cemiplimab Monotherapy of Patients with Brain Metastases from Advanced NSCLC with PD-L1 ≥50%

In the EMPOWER-Lung 1 (NCT03088540) phase III study, first-line cemiplimab monotherapy demonstrated superior median OS and progression-free survival (PFS), and produced higher objective response rates (ORRs) and longer duration of response (DOR) versus investigator's choice chemotherapy in patients with advanced NSCLC and PD-ligand 1 (PD-L1) ≥50% (NCCN, 2021). EMPOWER-Lung 1 included a notable proportion of patients with brain metastases (˜12% in the PD-L1 ≥50% populations).

This example provides results from the EMPOWER-Lung 1 study of cemiplimab monotherapy as first-line (1L) treatment of patients with brain metastases from advanced NSCLC with programmed cell death-ligand (PD-L1) ≥50%.

Background: In the phase 3 EMPOWER-Lung 1 study, cemiplimab monotherapy provided significant survival benefit and a better safety profile as compared to chemotherapy in patients with advanced NSCLC and PD-L1 expressed in >50% of tumor cells. EMPOWER-Lung 1 included patients with brain metastases at baseline who are typically underrepresented in clinical trials of first-line programmed cell death-1 (PD-1)/PD-L1 inhibitors. This study presents subgroup analysis of patients with brain metastasis from EMPOWER-Lung 1.

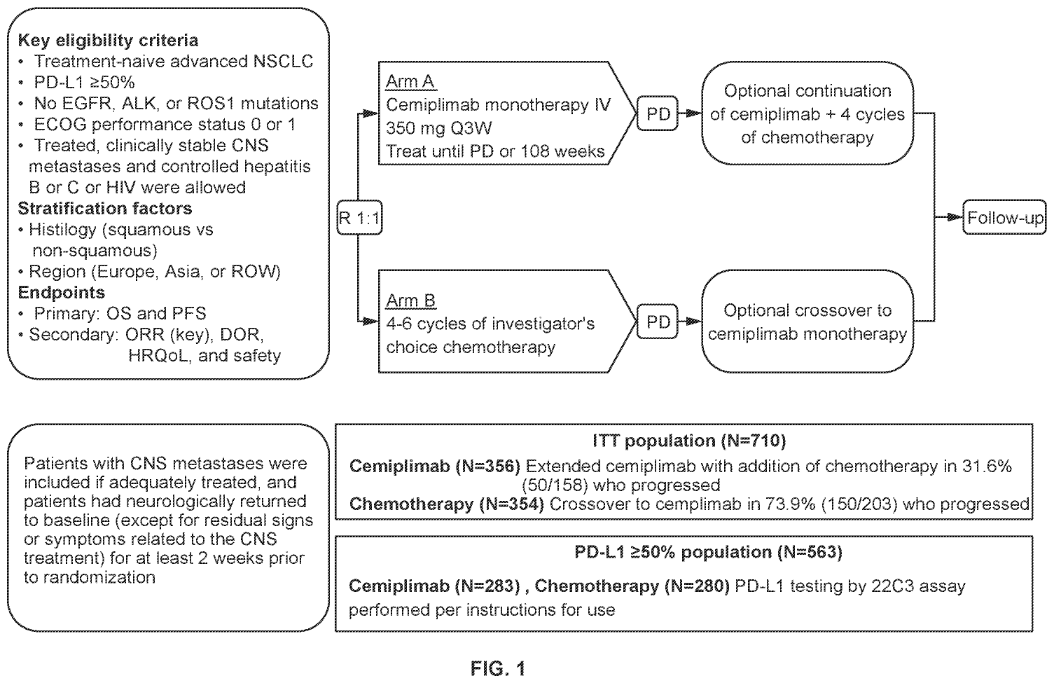

Methods: The design of this study is illustrated in FIG. 1. Patients were randomized 1:1 to cemiplimab 350 mg IV every 3 weeks or investigator's choice of chemotherapy (NCT03088540). Patients with treated, clinically stable brain metastases (radiological stability not required) were eligible to enroll and are the focus of this subgroup analysis from the PD-L1 ≥50% population (n=563) of the EMPOWER-Lung 1 study.

Tumor response to therapy was assessed according to RECIST 1.1, measured by CT or MRI. Radiographic tumor assessments were done every 3 cycles, at week 9, and every 9 weeks thereafter until disease progression. The present subgroup analysis focuses on patients with brain metastases from the PD-L1 ≥50% population. All patients received local therapy and needed to be neurologically stable before joining the trial. In patients with a known history of treated brain metastases at baseline, CT or MRI of the brain with contrast (unless contraindicated) was performed at screening (except if performed within 60 days prior to screening). Additional sites of known disease (including CNS) were also imaged at screening. During the treatment and follow-up period, surveillance imaging of the brain was performed every 18 weeks for year 1, and every 24 weeks for year 2, or sooner if clinically indicated.

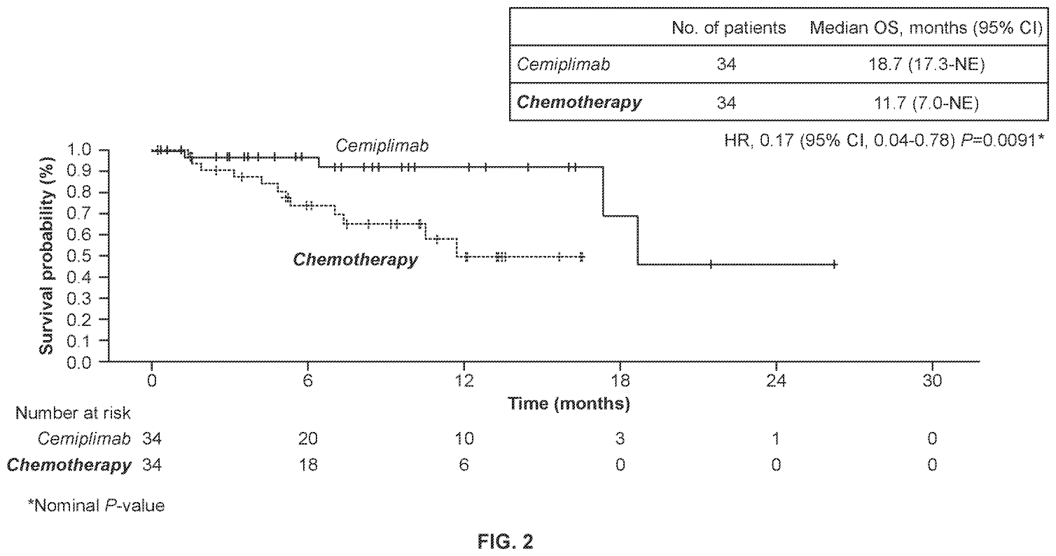

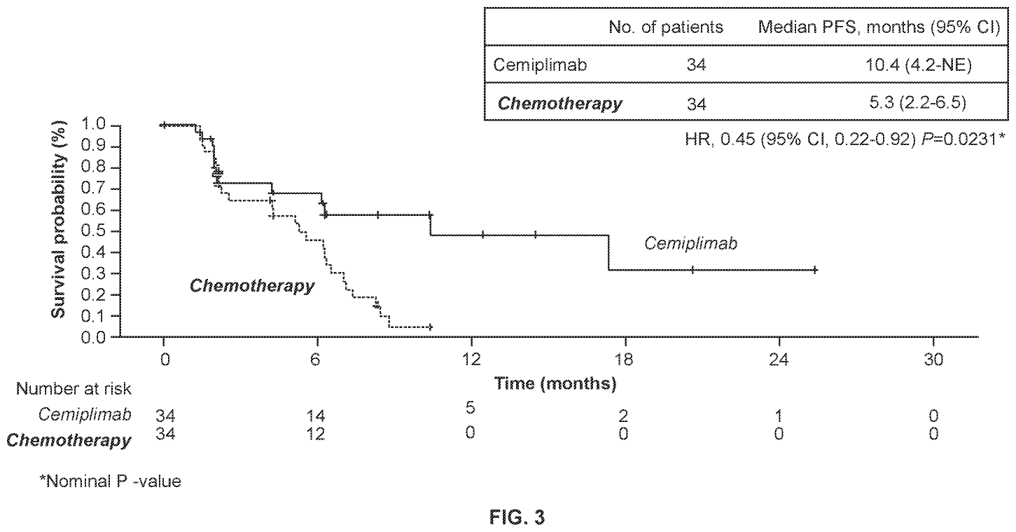

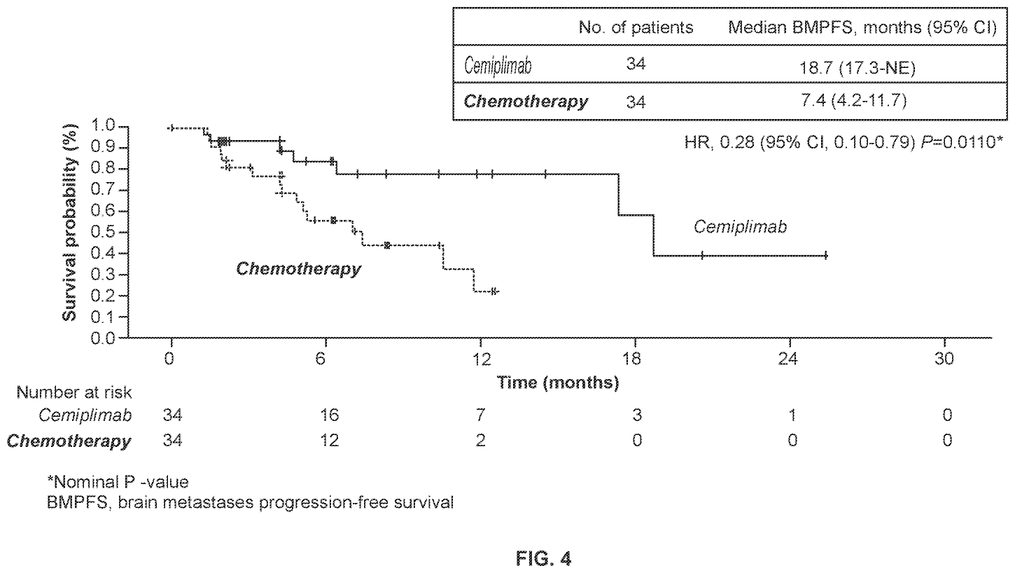

Results: A total of 68 of 563 (12.1%) cases had treated stable brain metastases at time of randomization. Patients were evenly distributed between cemiplimab (n=34) and chemotherapy (n=34), with similar median duration of follow-up. Baseline characteristics (Table 4) were generally similar; median (range) age: 60.0 (45-76) as compared to 62.0 (48-77); male: 97.1% as compared to 85.3%; and non-squamous histology: 85.3% as compared to 76.5%; between cemiplimab as compared to chemotherapy, respectively. Tumor responses were superior with cemiplimab versus chemotherapy (Table 5). Per independent review committee, median overall survival (OS, 18.7 as compared to 11.7 months), median progression-free survival (PFS, 10.4 as compared to 5.3 months), and objective response rate (ORR, 41.2% as compared to 8.8%) were superior with cemiplimab as compared to chemotherapy (Table 5; FIGS. 2-3). After baseline, central nervous system (CNS) intracranial disease progression occurred in two (5.9%) patients with cemiplimab as compared to four (11.8%) patients with chemotherapy. Intracranial disease progression due to a new tumor occurred in one (2.9%) patient with cemiplimab versus four (11.8%) patients with chemotherapy. Intracranial disease progression due to an existing tumor occurred in two (5.9%) patients with cemiplimab versus one (2.9%) patient with chemotherapy. Extra-CNS disease progression occurred in 9 (26.5%) patients with cemiplimab as compared to as compared to 15 (44.1%) patients with chemotherapy. Median KM estimation of brain metastasis PFS was superior with cemiplimab versus chemotherapy (FIG. 4).

TABLE 4
Patient Demographics And Baseline Characteristics

| | Cemiplimab | | Chemotherapy | |
|---|---|---|---|---|
| | (n = 34) | | (n = 34) | |
| Median age, years (range) | 60.0 | (45.0-76.0) | 62.0 | (48.0-77.0) |
| ≥65 years, n (%) | 9 | (26.5) | 12 | (35.3) |
| Male, n (%) | 33 | (97.1) | 29 | (85.3) |
| ECOG performance status, n (%) | | | | |
| 0 | 10 | (29.4) | 5 | (14.7) |
| 1 | 24 | (70.6) | 29 | (85.3) |
| Smoker status, n (%) | | | | |
| Current smoker | 7 | (20.6) | 12 | (35.3) |
| Past smoker | 27 | (79.4) | 22 | (64.7) |
| Histology, n (%) | | | | |
| Non-squamous | 5 | (14.7) | 8 | (23.5) |
| Squamous | 29 | (85.3) | 26 | (76.5) |
| Metastatic sites, n (%) | | | | |
| Lung | 18 | (52.9) | 26 | (76.5) |
| Liver | 4 | (11.8) | 5 | (14.7) |
| Bone | 4 | (11.8) | 7 | (20.6) |
| Adrenal | 9 | (26.5) | 6 | (17.6) |
| Lymph nodes, intrathoracic | 24 | (70.6) | 27 | (79.4) |
| Lymp nodes, other | 6 | (17.6) | 4 | (11.8) |
| Biochemical parameters, mean (SD) | | | | |
| Albumin, g/L | 38.7 | (4.9) | 38.8 | (5.4) |
| Alanine aminotransferase, IU/L | 24.5 | (21.5) | 25.4 | (23.7) |
| Aspartate aminotransferase, IU/L | 18.3 | (7.1) | 21.1 | (13.4) |
| Lactate dehydrogenase, IU/L | 242.0 | (94.7) | 282.5 | (113.0) |
| Median, duration of treatment | 24.0 | (11.9-45.0) | 13.4 | (9.3-21.1) |
| exposure, weeks (IQR) | | | | |
| Median duration of follow-up, weeks (IQR) | 9.2 | (3.7-16.3) | 9.3 | (6.1-13.3) |

IQR, interquartile range; IU/L, international units per liter; SD, standard deviation.

TABLE 5
Clinical Outcomes in Patients With Advanced NSCLC and Brain Metastases

| | Cemiplimab (n = 34) | | Chemotherapy (n = 34) | |
|---|---|---|---|---|
| Median duration of follow-up, weeks (IQR) | 9.2 | (3.7-16.3) | 9.3 | (6.1-13.3) |
| OS, median, months (95% CI) | 18.7 | (17.3-NE) | 11.7 | (7.0-NE) |
| HR (95% CI) | 0.17 (0.04-0.76); P = 0.0091 | | | |
| PFS, median, months (95% CI) | 10.4 | (4.2-NE) | 5.3 | (2.2-6.5) |
| HR (95% CI) | 0.45 (0.22-0.92); P = 0.0231 | | | |
| ORR, %, (95% CI) | 41.2% | (24.6-59.3) | 8.8% | (1.9-23.7) |
| Odds ratio (95% CI) | 6.9 (1.7-27.8); P = 0.0034* | | | |
| Best overall tumor response, n (%) | | | | |
| Complete response | 3 | (8.8) | 0 | |
| Partial response | 11 | (32.4) | 3 | (8.8) |
| Stable disease | 9 | (26.5) | 18 | (52.9) |
| Non-complete response/non-partial | — | | — | |
| disease | | | | |
| Progressive disease | 7 | (20.6) | 9 | (26.5) |
| Non-evaluable | 4 | (11.8) | 4 | (11.8) |

HR, hazard ratio; IQR, inter-quartile range; NE, not evaluable.
*Nominal P-value.
In patients with brain metastases at baseline, the brain was a non-target lesion.

Patient Reported Outcomes (PROs): Post-hoc exploratory analyses were conducted to evaluate PROs in this subgroup of aNSCLC patients with PD-L1 ≥50% and clinically stable treated brain metastases at baseline. PROs were assessed at baseline and day 1 of each treatment cycle for the first 6 cycles, and then on day 1 of every third cycle using EORTC QLQ-C30 and QLQ-LC13 questionnaires. Higher scores indicate better functioning, global health status (GHS)/quality of life (QoL), or worse symptom severity. Mixed model for repeated measures analyses were performed to compare overall change from baseline scores between the two treatment arms, while controlling for baseline characteristics. The baseline PRO scores of the cemiplimab arm were broadly similar with the chemotherapy arm. A statistically significant overall change from baseline in GHS/QOL favoring cemiplimab versus chemotherapy (9.35; 95% CI, 2.24, 16.45; p=0.0110) was observed. Cemiplimab also resulted in a statistically significant favorable difference in overall change from baseline in role functioning (8.59; 95% CI, 0.16, 17.01; p=0.0459), emotional functioning (7.27; 95% CI, 1.86, 12.69; p=0.0095), and symptoms of fatigue (−8.19; 95% CI, −15.40, −0.98; p=0.0268) and appetite loss (−7.43; 95% CI, −14.48, −0.38; p=0.0393). When comparing between arms, no analyses yielded statistically significant PRO results favoring chemotherapy for any QLQ-C30 or QLQ-LC13 scale.

Conclusions: 1L cemiplimab monotherapy improved OS, PFS, and ORR as compared to chemotherapy, in patients with advanced NSCLC with PD-L1 ≥50%, who had clinically stable brain metastases at baseline. Cemiplimab monotherapy represents a surprisingly effective treatment option for this subgroup of patients. Additionally, cemiplimab resulted in significantly favorable overall change from baseline in GHS/QOL, role and emotional functioning, and fatigue and appetite loss symptoms compared with chemotherapy in this subgroup of patients. PRO results further support the favorable benefit-risk profile of first-line cemiplimab versus chemotherapy in advanced NSCLC with PD-L1 ≥50% and clinically stable brain metastases.

### Example 3: Results of Clinical Trial of Cemiplimab Monotherapy for the Treatment of Advanced NSCLC with PD-L1 ≥50%

This example provides results from an EMPOWER-Lung 1 study, including patient-reported symptoms, function, and quality of life (QoL) in patients treated with cemiplimab monotherapy for first-line treatment of advanced NSCLC with PD-L1 expressed in ≥50% of tumor cells.

Background: Cemiplimab, a PD-1 inhibitor, improved survival and progression free survival as compared to platinum doublet chemotherapy (chemo) in patients with advanced NSCLC and PD-L1 expression ≥50% in the EMPOWER-Lung 1 phase 3 study (NCT03088540). An objective of the study was to evaluate the effects of cemiplimab on symptom burden, functioning, and QoL in patients with NSCLC. Since patients with advanced NSCLC have a high symptom burden that adversely impacts QoL and functioning, these outcomes were evaluated as secondary endpoints in the clinical trial. Disease-related symptoms of importance to patients include shortness of breath, cough, fatigue, and pain, and chemotherapy-related symptoms include neuropathy and sore mouth.

Methods: EMPOWER-Lung 1 was a multicenter, open-label, randomized, controlled, Phase III, pivotal clinical trial of cemiplimab in adults (≥18 years) with: (i) histologically or cytologically confirmed Stage IIIB/IIIC or Stage IV squamous or non-squamous NSCLC with PD-L1 expressed in ≥50% of tumor cells; (ii) ECOG performance status ≤1; (iii) adequate organ and bone marrow function; and (iv) adequately treated clinically stable brain metastases (historically underrepresented in clinical trials) were allowed to enroll.

A total of 710 patients were randomized to IV cemiplimab 350 mg Q3W for up to 36 treatment cycles (n=356) or platinum doublet chemotherapy determined prior to randomization for 4 to 6 treatment cycles (n=354). At baseline (BL) and day 1 of each treatment cycle (C) for the first 6 cycles, and then on day 1 of every 3 cycles to C15, patients were administered the EORTC core questionnaire (QLQ-C30) and its lung cancer specific module (QLQ-LC13) to assess symptoms, functioning, and Global Health Status (GHS)/QoL. In the intent-to-treat population, mixed-effects repeated measures models (mixed-model repeat measures, MMRM) were used to estimate least squares (LS) mean change from BL on all scales. Follow-up assessment was conducted 14 to 30 days after the last study treatment administration if a patient discontinued, or 14 to 30 days (±7 days) after the last cycle visit if the patient completed treatment.

The QLQ-C30 assesses the patient-reported GHS/QOL scale and across functioning (physical, role, cognitive, emotional, and social) and symptoms (fatigue, pain, nausea/vomiting, dyspnea, insomnia, appetite loss, constipation, diarrhea, and financial difficulties) over the past week among cancer patient populations. Scores range from 0 to 100; high scores on functional domains and low scores on symptoms reflect better outcomes. The QLQ-LC13 is a patient-reported outcome measure that assesses lung cancer disease- and treatment-specific symptoms during the past week. Scores range from 0 to 100; higher scores reflect higher symptom levels. A change in QLQ-C30 and QLQ-LC13 scores of at least 10 points absolute value from baseline was considered clinically meaningful. MMRM analysis was used to estimate LS mean change from baseline on all scales among patients with a baseline and at least one post-baseline score.

The analysis included all cycles where at least 10 patients per arm were available and was truncated at C15 since <10 patients per arm were available. Kaplan-Meier (KM) survival analysis over the study period estimated time to definitive deterioration, defined as clinically meaningful worsening (i.e., >10 points) from baseline observed at all subsequent time points or patient withdrawal after worsening. Hazard ratios (HRs) with 95% confidence intervals (95% CIs) were derived from the survival analyses to determine the likelihood of definitive deterioration. Analyses were based on the intention-to-treat (ITT) population consisting of all randomized patients. Analyses were also conducted in a modified ITT population (mITT), which was prespecified to include patients with PD-L1 ≥50% per assay according to manufacturer's instructions for use.

Results: Baseline patient characteristics of the ITT population were similar between treatment groups (Table 6). The majority of patients (84%) had metastatic disease. Baseline characteristics of the mITT population (n=563) were similar to those of the ITT population.

TABLE 6
Baseline Patient Characteristics*

| | Cemiplimab | | Chemotherapy | |
|---|---|---|---|---|
| | (n = 356) | | (n = 354) | |
| Age, median (IQR), years | 63 | (58-69) | 64 | (57-69) |
| ≥65 years | 156 | (44) | 164 | (46) |
| Male | 312 | (88) | 294 | (83) |
| Region of enrollment | | | | |
| Europe | 275 | (77) | 278 | (79) |
| Asia | 39 | (11) | 38 | (11) |
| Rest of world | 42 | (12) | 38 | (11) |
| ECOG performance status score | | | | |
| 0 | 96 | (27) | 96 | (27) |
| 1 | 260 | (73) | 258 | (73) |
| Smoking status | | | | |
| Current smoker | 133 | (37) | 120 | (34) |
| Past smoker | 223 | (63) | 234 | (66) |
| Histology | | | | |
| Squamous | 59 | (45) | 152 | (43) |
| Non-squamous | 197 | (55) | 202 | (57) |
| Brain metastases | 44 | (12) | 39 | (11) |
| Stage at screening | | | | |
| Locally advanced | 63 | (18) | 52 | (15) |
| Metastatic | 293 | (82) | 302 | (85) |
| Previous systemic | 4 | (1) | 7 | (2) |
| neoadjuvant therapy | | | | |
| Previous systemic | 9 | (3) | 15 | (4) |
| adjuvant therapy | | | | |

*Values are number (percent) of patients unless otherwise indicated.
IQR, interquartile range.

Baseline scores on the QLQ-C30 showed moderate to high levels of functioning and low symptom burden in both the ITT and mITT populations. The highest symptom burdens were reported for fatigue and coughing. Cemiplimab-treated patients had lower likelihood of definitive deterioration on key symptoms of dyspnea, cough, pain in chest, pain in other body parts, fatigue, nausea/vomiting, appetite loss, constipation, and diarrhea, as compared to chemotherapy (all P<0.05). Treatment-related symptoms of peripheral neuropathy and alopecia had a lower likelihood of definitive deterioration with cemiplimab as compared to chemotherapy (both P<0.05). Cemiplimab resulted in significantly greater improvements as compared to chemotherapy on GHS/QOL and all functioning scales and reduced the likelihood of definitive deterioration to a statistically significant degree as indicated by HR<1 (Table 7).

TABLE 7

| | Difference in LS mean | HR (95% CI) |
|---|---|---|
| | change from BL | for definitive |
| | (95% CI)^{a} | deterioration |
| GHS/QoL | 5.4 (2.7-8.1)* | 0.73 (0.52-1.03) |
| Physical functioning | 4.2 (1.6-6.7)* | 0.59 (0.42-0.84)* |
| Role functioning | 3.3 (0.2-6.3)* | 0.65 (0.46-0.92)* |
| Emotional functioning | 3.4 (1.0-5.9)* | 0.49 (0.30-0.79)* |
| Cognitive functioning | 2.5 (0.4-4.5)* | 0.64 (0.44-0.93)* |
| Social functioning | 5.2 (2.5-7.9)* | 0.50 (0.35-0.72)* |

^{a}Cemiplimab minus chemo
*P < .05

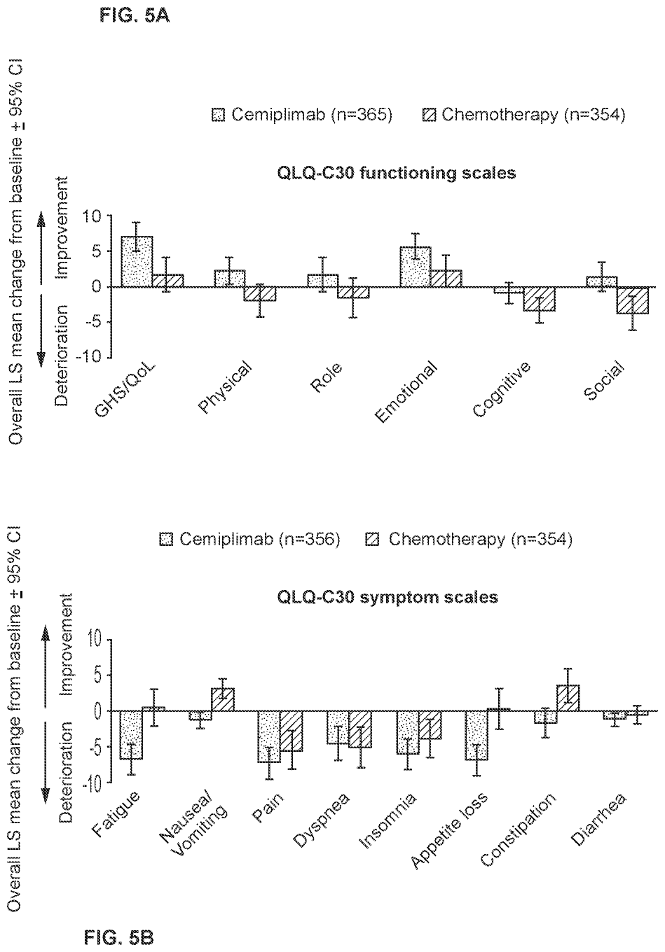

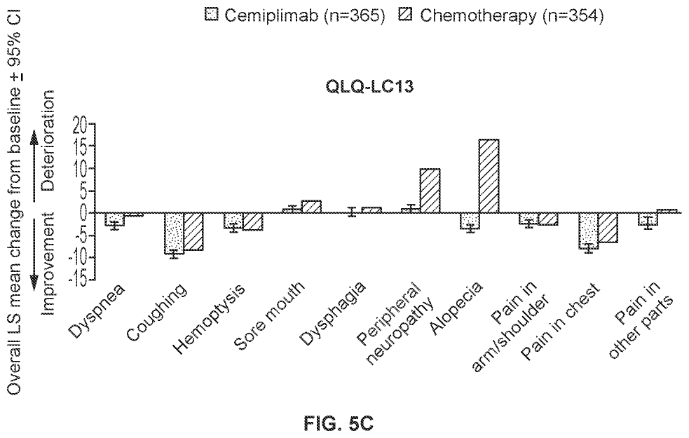

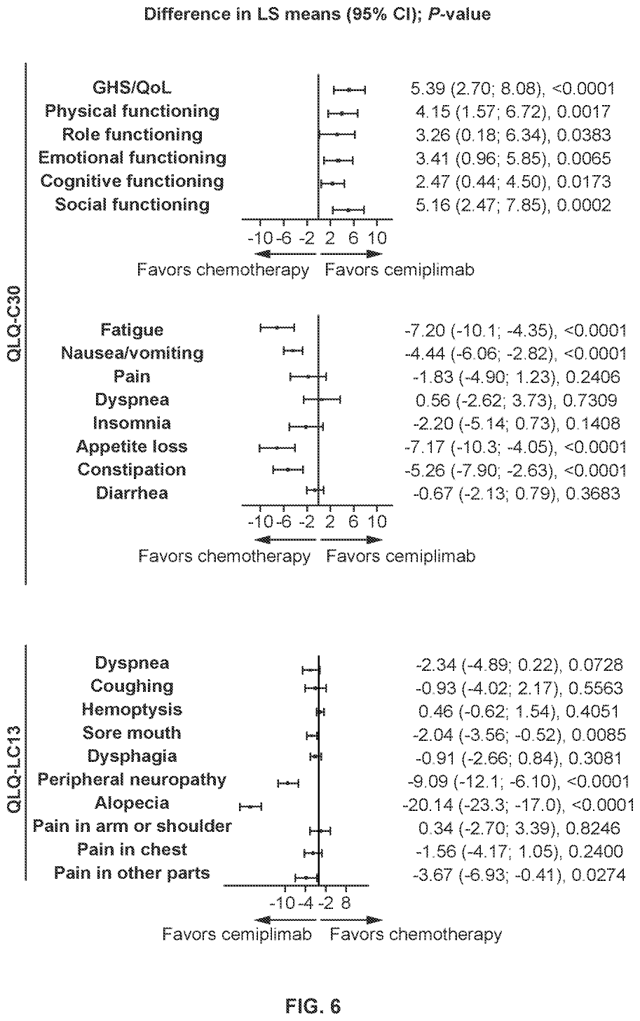

As shown in FIGS. 5A, 5B, 5C, and 6, MMRM analysis showed that the overall change from baseline across the first year of treatment consistently favored cemiplimab with significant improvements versus chemotherapy for GHS/QOL, functioning (all five scales), and key symptoms including disease-related symptoms of fatigue and pain in other body parts, and treatment-related symptoms of alopecia, appetite loss, constipation, nausea/vomiting, peripheral neuropathy, and sore mouth. Similar results were observed in the mITT population.

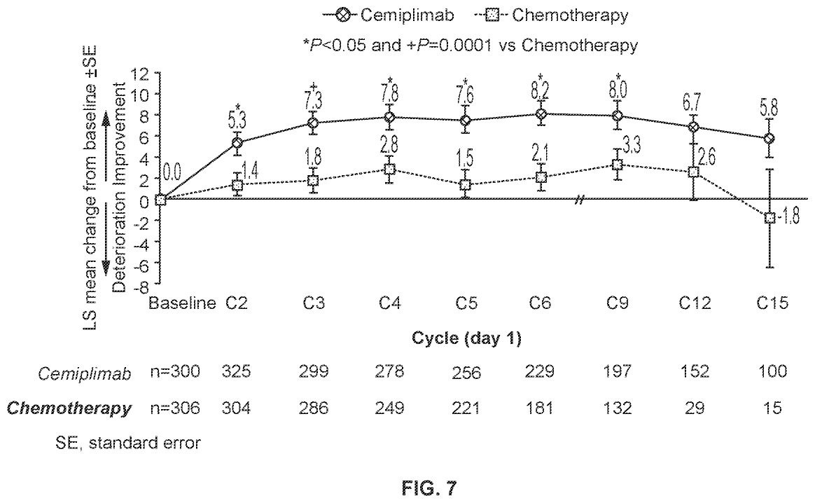

MMRM analysis showed statistically significant improvements in GHS/QOL with cemi-plimab versus chemotherapy were observed as early as C2 and were maintained to C15 (FIG. 7). LS mean change (SE) from baseline across all timepoints was 7.1 (1.0) for cemiplimab as compared to 1.7 (1.2) for chemotherapy (P<0.0001).

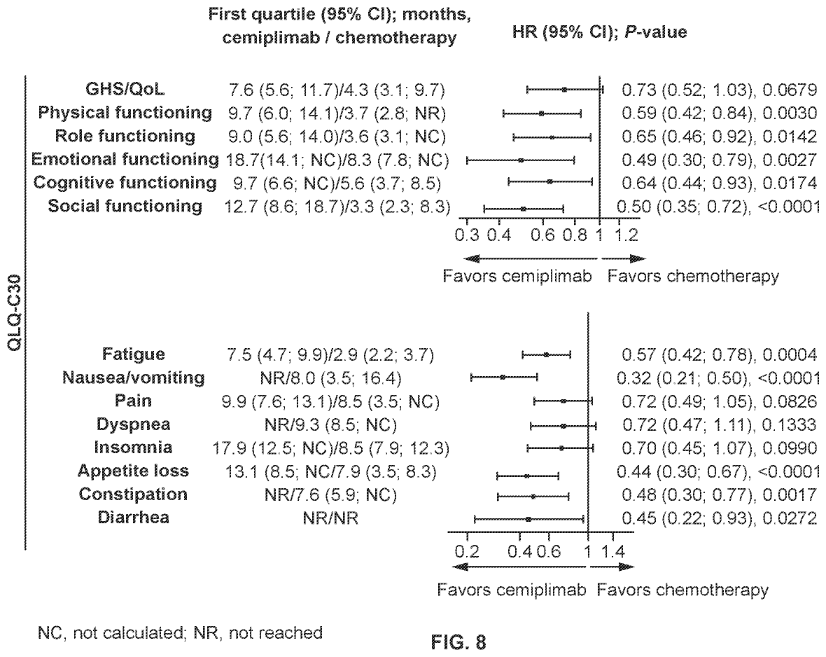

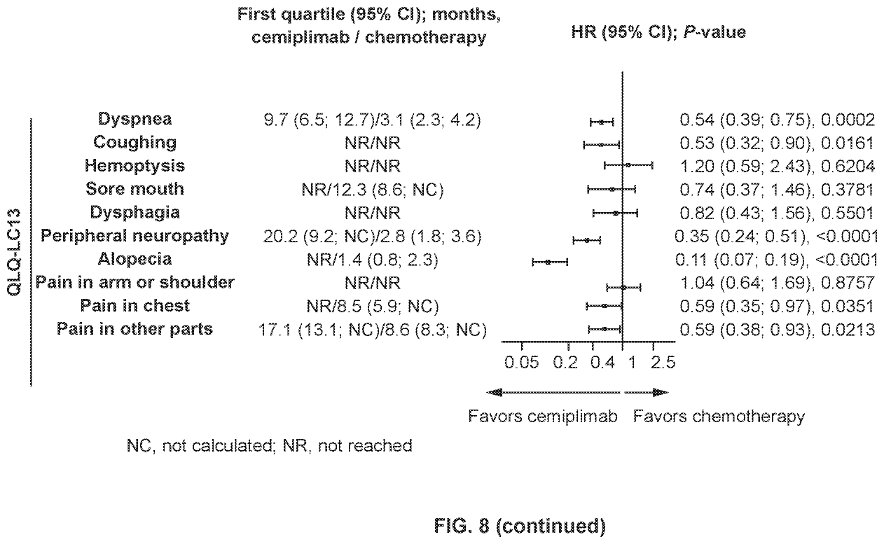

Cemiplimab reduced the risk of definitive deterioration in GHS/QOL and all functioning scales, although GHS/QOL did not achieve statistical significance (FIG. 8). Although the median time to definitive deterioration was not reached for most symptoms, cemiplimab-treated patients had a significantly lower risk of definitive deterioration versus chemotherapy over the study period on key symptoms of dyspnea, cough, pain in chest, pain in other body parts, fatigue, nausea/vomiting, appetite loss, constipation, and diarrhea versus chemotherapy (all P<0.05) (FIG. 8). Treatment-related symptoms of peripheral neuropathy and alopecia also had a lower risk of definitive deterioration with cemiplimab versus chemotherapy (both P<0.05).

These results indicate that cemiplimab delayed the time to definitive deterioration in GHS/QOL, functioning, and key symptoms. Similar trend and magnitude of effects were observed in the mITT population.

Conclusions: In patients with advanced NSCLC and PD-L1 expression ≥50%, cemiplimab provided statistically significant improvements in GHS/QOL, functioning, and most symptoms as compared to chemotherapy over the first year of treatment as measured by the EORTC QLQ-C30 and QLC-LC13. Cemiplimab delayed worsening of key lung cancer- and treatment-related symptoms, functioning, and GHS/QOL as indicated by lower likelihood of definitive deterioration in these outcomes over the study period. Thus, cemiplimab monotherapy provided a surprisingly superior treatment option for patients with advanced NSCLC and PD-L1 expression ≥50%.

### Example 4: Results of First-Line Cemiplimab in Patients with Locally Advanced NSCLC

This example provides results from an EMPOWER-Lung 1 study of a subgroup of patients with laNSCLC treated with cemiplimab.

Background: There is paucity of prospective clinical data investigating the use of anti-PD-1 agents in patients with laNSCLC who are not candidates for definitive concurrent chemoradiotherapy. In the Phase 3 EMPOWER-Lung 1 Study (NCT03088540), cemiplimab, an anti-PD-1 antibody, demonstrated improved overall survival (OS) and progression-free survival (PFS) in patients with advanced NSCLC and PD-L1 ≥50% vs platinum-doublet chemotherapy. This study allowed the enrollment of patients with laNSCLC, in addition to those with metastatic disease, providing the largest prospective randomized evidence of first-line (1L) anti-PD-1 monotherapy in this patient population. This example describes a subgroup analysis of patients with laNSCLC from the PD-L1 ≥50% population in EMPOWER-Lung 1.

Methods: In EMPOWER-Lung 1, patients were randomized 1:1 to cemiplimab 350 mg intravenous every 3 weeks or investigator's choice of platinum-doublet chemotherapy. Patients with locally advanced non-small cell lung cancer (laNSCLC) were those with Stage IIIB/IIIC disease who were not candidates for definitive concurrent chemoradiotherapy.

Results: In the PD-L1 ≥50% population of EMPOWER-Lung 1 (n=563), 87 (15.5%) patients had laNSCLC; cemiplimab (n=45) and chemotherapy (n=42). In the total laNSCLC population (n=87), median (range) age was 63.0 (31.0-81.0); male: 86.2%; non-squamous histology: 36.8%; Stage IIIB cancer: 79.3%; and Stage IIIC cancer: 20.7%. At a median follow up of 11.6 months (inter-quartile range 7.2-18.2 months), cemiplimab provided significantly better PFS vs chemotherapy (see Table 8). OS results were also improved but did not reach statistical significance. Objective response rates (ORR) and Kaplan-Meier estimated median duration of response (DOR) were also numerically improved with cemiplimab vs chemotherapy.

TABLE 8
Clinical Endpoint Results in Patients With laNSCLC

| | Cemiplimab | | Chemotherapy | |
|---|---|---|---|---|
| | (n = 45) | | (n = 42) | |
| Median duration of follow-up, | 12.2 | (7.2-21.8) | 11.6 | (7.8-17.4) |
| months (IQR) | | | | |
| PFS, median, months | 8.4 | (4.5-15.3) | 6.2 | (4.6-6.6) |
| (95% CI) | | | | |
| HR (95% CI) | 0.5 (0.3-0.9); P = 0.0153 | | | |
| OS, median, months (95% CI) | NE | (17.7-NE) | 15.5 | (9.6-NE) |
| HR (95% CI) | 0.5 (0.2-1.1); P = 0.0875 | | | |
| ORR, n, (95% CI) | 20.0 | (29.6-60.0) | 13.0 | (17.6-47.1) |
| CR, n, (%) | 1 | (2.2) | 1 | (2.4) |
| PR, n, (%) | 19 | (42.2) | 12 | (28.6) |
| KM estimated DOR, median, | 12.5 | (6.4-21.0) | 6.2 | (3.4-8.5) |
| months (95% CI)^{†} | | | | |

†Cemiplimab n = 20 and chemotherapy n = 13.
CR, complete response; PR, partial response; HR, hazard ratio; IQR, inter-quartile range; KM, Kaplan-Meier; NE, not evaluable.

Conclusion: In patients with laNSCLC and with PD-L1 ≥50%, 1L cemiplimab monotherapy demonstrated a significant improvement in PFS, numerically longer OS, and improved ORR and DOR versus chemotherapy. These results support clinical benefit provided by cemiplimab 1L monotherapy for patients with laNSCLC with PD-L1 ≥50%.

### Example 5: Clinical Benefit of First-Line Cemiplimab in laNSCLC Patient Subgroup

This Example Provides Results from an EMPOWER-Lung 1 Study (NCT03088540) of a subgroup of patients with laNSCLC and PD-L1 ≥50% and without EGFR, ALK or ROS1 driver mutations.

Study Design: Key eligibility criteria included: (i) treatment-naïve advanced NSCLC; (ii) PD-L1 ≥50%; (iii) no EGFR, ALK, or ROS1 mutations; (iv) ECOG PS 0 or 1; and (v) treated, clinically stable CNS metastases and controlled hepatitis B or C or HIV were allowed. Stratification factors included: histology (squamous vs non-squamous) and region (Europe, Asia, or ROW). Primary endpoints: OS and PFS. Secondary endpoints: ORR (key), DOR, HRQoL, and safety. The patients had locally advanced NSCLC, including those with Stage IIIB/IIIC disease who were not candidates for treatment with definitive concurrent chemoradiotherapy or had recurred after initial treatment with concurrent chemoradiotherapy.

Arm A patients received cemiplimab monotherapy IV, 350 mg Q3W, treated until progressive disease (PD) or 108 weeks; if PD, optional continuation of cemiplimab in combination with 4 cycles of chemotherapy. Arm B patients received 4-6 cycles of investigator's choice chemotherapy; if PD, optional crossover to cemiplimab monotherapy. PD-L1 ≥50% population (N=563): cemiplimab (n=283), chemotherapy (n=280). PD-L1 testing by 22C3 assay performed. Patient demographics and baseline characteristics are provided in Table 9. In the PD-L1 ≥50% population of EMPOWER-Lung 1 (n=563), 87 (15.5%) patients had locally advanced NSCLC: cemiplimab (n=45) and chemotherapy (n=42).

TABLE 9
Patient Demographics And Baseline Characteristics

| | Cemiplimab | | Chemotherapy | |
|---|---|---|---|---|
| | (n = 45) | | (n = 42) | |
| Age | | | | |
| Median (range) | 63.0 | (31.0-79.0) | 63.5 | (43.0-81.0) |
| ≥65 years, n (%) | 17 | (37.8) | 19 | (45.2) |
| Male sex, n (%) | 41 | (91.1) | 34 | (81.0) |
| ECOG PS, n (%) | | | | |
| 0 | 16 | (35.6) | 13 | (31.0) |
| 1 | 29 | (64.4) | 29 | (69.0) |
| Smoking status, n (%) | | | | |
| Patients with active tobacco | 22 | (48.9) | 16 | (38.1) |
| use | | | | |
| Patients with history of active | 23 | (51.1) | 26 | (61.9) |
| tobacco use | | | | |
| Histology, n (%) | | | | |
| Squamous | 27 | (60.0) | 28 | (66.7) |
| Non-squamous | 18 | (40.0) | 14 | (33.3) |
| Cancer stage at screening, | | | | |
| n (%) | | | | |
| IIIB | 36 | (80.0) | 33 | (78.6) |
| IIIC | 9 | (20.0) | 9 | (21.4) |
| PD-L1 expression levels, | | | | |
| n (%) | | | | |
| ≥90% | 18 | (40.0) | 18 | (42.9) |
| >60 to <90% | 11 | (24.4) | 10 | (23.8) |
| ≥50 to ≤60% | 16 | (35.6) | 14 | (33.3) |

Patient exposure to treatment and duration of follow-up is shown in Table 10.

TABLE 10
Patient Exposure To Treatment And Duration Of Follow-Up

| | Cemiplimab | | Chemotherapy | |
|---|---|---|---|---|
| | (n = 45) | | (n = 42) | |
| Median duration of | 30.7 | (11.9-43.6) | 17.7 | (13.0-20.0) |
| exposure, weeks (IQR)^{†} | | | | |
| Median duration of | 12.2 | (7.2-21.8) | 11.6 | (7.8-17.4) |
| follow-up, months (IQR) | | | | |
| Duration of follow-up, | | | | |
| months, n (%) | | | | |
| ≥6 | 35 | (77.8) | 32 | (76.2) |
| ≥12 | 23 | (51.1) | 19 | (45.2) |
| ≥18 | 13 | (28.9) | 9 | (21.4) |
| ≥24 | 8 | (17.8) | 5 | (11.9) |

| | | |
|---|---|---|
| ≥30 | 0 | 0 |

^{†}For the duration of exposure analysis, one patient in the chemotherapy arm was not available for analysis (n = 41). IQR, interquartile range.

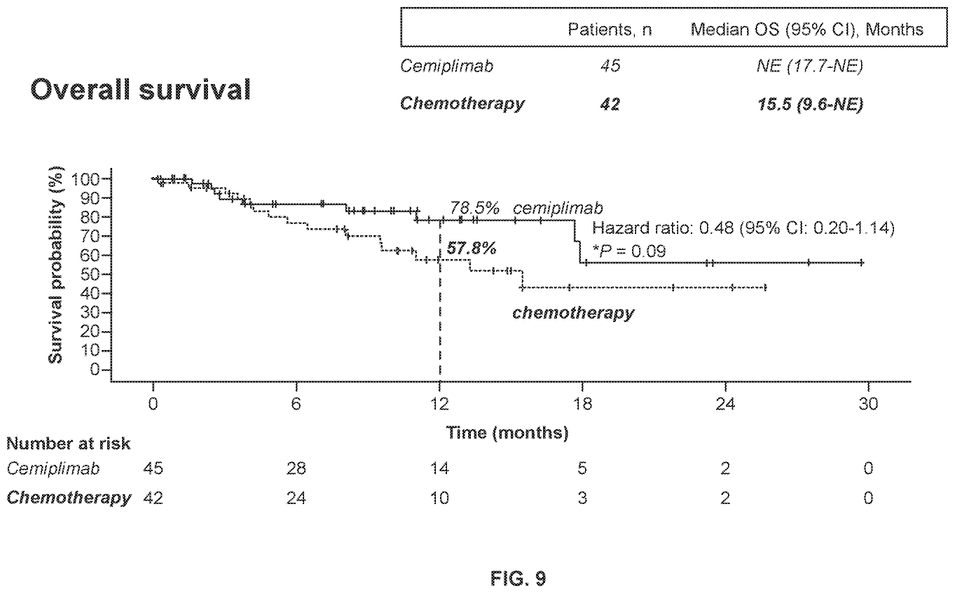

As shown in FIG. 9, median OS was not yet reached for cemiplimab patients, whereas median OS was 15.5 months for chemotherapy patients. Additionally, 12-month OS was achieved for 78.5% of cemiplimab patients as compared to 57.8% of chemotherapy patients. Cemiplimab demonstrated a numerically longer overall survival, resulting in a hazard ratio of 0.48. Thus, cemiplimab treatment provided substantially improved overall survival as compared to chemotherapy.

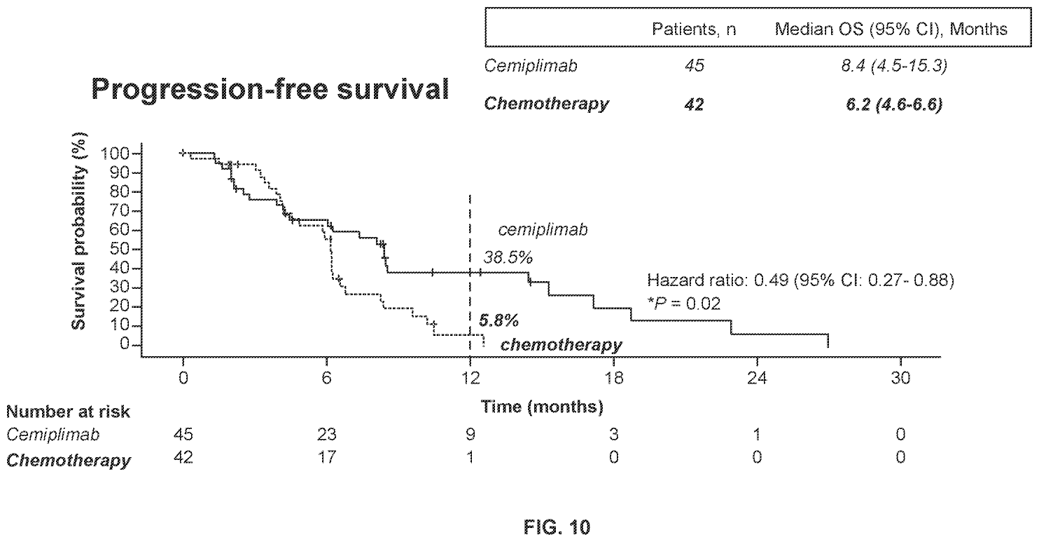

As shown in FIG. 10, there was a statistically significant improvement associated with cemiplimab as compared to chemotherapy. Specifically, median PFS was 8.4 months for cemiplimab patients, whereas median PFS was 6.2 months for chemotherapy patients. The hazard ratio was 0.49. Additionally, 12-month PFS was achieved for 38.5% of cemiplimab patients as compared to 5.8% of chemotherapy patients. Thus, cemiplimab treatment provided substantially improved progression-free survival as compared to chemotherapy.

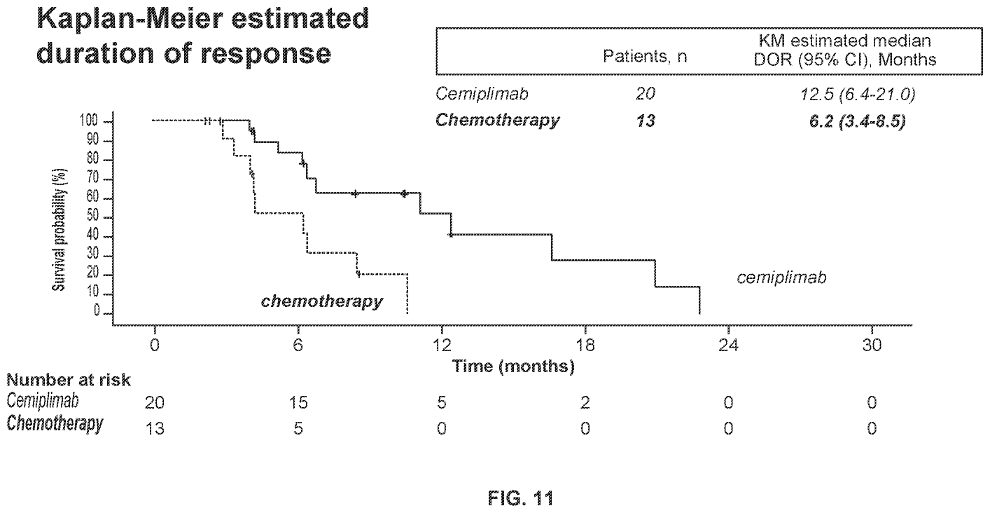

Duration of response was also numerically longer with cemiplimab. As shown in FIG. 11, KM estimated median DOR was 12.5 months for cemiplimab patients, whereas median DOR was 6.2 months for chemotherapy patients. Thus, cemiplimab treatment provided substantially improved duration of response as compared to chemotherapy.

The objective response rate was numerically higher with cemiplimab (44%) versus chemotherapy (31%); and more patients experienced a partial response as their best overall response with cemiplimab (Table 11).

TABLE 11
Tumor Response

| | |
|---|---|
| Cemiplimab | Chemotherapy |
| (n = 45) | (n = 42) |

| | | | | |
|---|---|---|---|---|
| ORR, n (%) | 20 | (44.4) | 13 | (31.0) |

| | | |
|---|---|---|
| 95% CI | (29.6-60.0) | (17.6-47.1) |

| | |
|---|---|
| Odds ratio (95% CI) | 1.8 (0.7-4.2); *P = 0.20 |

Best overall response, n (%)

| | | | | |
|---|---|---|---|---|
| CR | 1 | (2.2) | 1 | (2.4) |
| Partial response | 19 | (42.2) | 12 | (28.6) |
| Stable disease | 10 | (22.2) | 18 | (42.9) |

| | | | |
|---|---|---|---|
| Non-CR/Non-PD | 0 | 1 | (2.4) |

| | | | | |
|---|---|---|---|---|
| PD | 7 | (15.6) | 1 | (2.4) |
| Non-evaluable | 8 | (17.8) | 9 | (21.4) |

Conclusions: In this post-hoc subgroup analysis from EMPOWER Lung-1, patients with locally advanced NSCLC and with PD-L1 ≥50% demonstrated improved survival benefits with first-line cemiplimab monotherapy compared with chemotherapy, including: significantly improved PFS with a 51% reduction in the risk of disease progression with cemiplimab vs chemotherapy (HR, 0.49; 95% CI, 0.27-0.88; * P=0.02); numerically longer OS (12-month OS rate: 78.5% vs 57.8% for cemiplimab versus chemotherapy); and numerically better ORR (44.4% vs 31.0%) and longer DOR (median 12.5 versus 6.2 months) for cemiplimab versus chemotherapy. These results demonstrate a significant benefit provided by cemiplimab first-line monotherapy for patients with locally advanced NSCLC and with PD-L1 ≥50% without EGFR, ALK, or ROS1 driver mutations who are not candidates for definitive concurrent chemoradiotherapy.

### Example 6: Hospitalization and Supportive Care Medication Utilization in Patients with Advanced NSCLC

This example provides results from an EMPOWER-Lung 1 Study (NCT03088540) showing a statistically significant and clinically meaningful improvement in overall survival (hazard ratio, 0.57; 95% confidence interval, 0.42, 0.77; P=0.0002) and progression-free survival (hazard ratio, 0.54; 95% confidence interval, 0.43, 0.68; P<0.0001), and favorable patient-reported outcomes with cemiplimab monotherapy versus physician's choice of platinum-doublet chemotherapy in patients with advanced NSCLC (aNSCLC) with PD-L1 ≥50%. Hospitalization and supportive care medication (SCM) utilization was evaluated while accounting for cemiplimab or chemotherapy treatment-emergent periods in EMPOWER-Lung 1.

Methods: In the EMPOWER-Lung 1 safety population (while patients were on cemiplimab or chemotherapy), the following were compared between cemiplimab- and chemotherapy-treated patients: serious adverse event (SAE)-associated hospitalization rates (per 100 patient-years), number of patients with ≥1 platelet/red blood cell (PRBC) transfusions, and 11 types of SCM (opiate, antiemetic/antinauseant, antidiarrheal, appetite stimulant, bone disease treatment, anti-anemic, immunostimulant, antibacterial, antimycotic, antiviral, and corticosteroid) utilization ratios (total duration of each SCM type/treatment-emergent period, in 1000 days).

Results: EMPOWER-Lung 1 safety population consisted of 355 cemiplimab- and 342 chemotherapy-treated patients. Lower rates of SAE-associated hospitalization were observed with cemiplimab-versus chemotherapy-treated patients (33.2 vs 56.7 per 100 patient-years). One (0.3%) cemiplimab-treated patient versus 3 (0.9%) chemotherapy-treated patients had ≥1 platelet transfusions; 4 (1.1%) cemiplimab-treated patients versus 24 (7.0%) chemotherapy-treated patients had ≥1 PRBC transfusions. Nine of 11 SCM utilization ratios for cemiplimab-treated patients were lower than those for chemotherapy-treated patients (Table 12).

Conclusions: In EMPOWER-Lung 1, patients with aNSCLC treated with cemiplimab versus chemotherapy had lower SAE-associated hospitalization rates. Except for antidiarrheal and antiviral for systemic use, all other SCM utilization ratios were lower with cemiplimab. These results support the favorable clinical and patient-reported outcomes observed with cemiplimab-versus chemotherapy-treated patients in EMPOWER-Lung 1.

TABLE 12
Supportive Care Medication utilization in EMPOWER-Lung 1

| | SCM utilization ratio in 1000 days | | Difference in ratio |
|---|---|---|---|
| | CEMI | CHEMO | (CHEMO − CEMI) |
| SCM category | (n = 355) | (n = 342) | in 1000 days |
| Platelet transfusion | 0.01 | 0.05 | 0.04 |
| PRBC transfusion | 0.07 | 0.44 | 0.37 |
| Anti-anemic | 52.49 | 264.29 | 211.79 |
| Antidiarrheal | 10.57 | 3.92 | −6.65 |
| Antiemetic/antinauseant | 2.52 | 205.42 | 202.90 |
| Appetite stimulant | 5.13 | 25.62 | 20.49 |
| Bone disease treatment | 3.07 | 18.82 | 15.75 |
| Immunostimulant | 0.21 | 14.81 | 14.60 |
| Opiate | 70.90 | 149.94 | 79.04 |
| Antibacterial for systemic use | 22.34 | 38.71 | 16.37 |
| Antimycotic for systemic use | 0.87 | 2.36 | 1.49 |
| Antiviral for systemic use | 1.16 | 0.83 | −0.33 |
| Corticosteroid for systemic use | 26.71 | 171.78 | 145.06 |

CEMI, cemiplimab; CHEMO, chemotherapy; PRBC, platelet/red blood cell; SCM, supportive care medication.

### REFERENCES

1. Aaronson N K et al. J Natl Cancer Inst. 1993; 85:365-376.
2. Abrey L E. Clinical Investig. 2011; 1:1065-1068.
3. Ali A et al. Curr Oncol. 2013; 20: e300-306.
4. ALIMTA [package insert]. Indianapolis, IN: Eli Lilly and Company; 2013.
5. ALIMTA [SmPC]. Indianapolis, IN: Eli Lilly and Company; 2013.
6. American Cancer Society. Key Statistics for Lung Cancer.

https://www.cancer.org/cancer/lung-cancer/about/key-statistics.html. (2020)
7. Bergman et al., “The EORTC QLQ-LC13: a modular supplement to the EORTC Core Quality of Life Questionnaire (QLQ-C30) for use in lung cancer clinical trials. EORTC Study Group on Quality of Life,” Eur J Cancer. 1994; 30A(5):635-42.
8. Besse et al., 2nd ESMO Consensus Conference on Lung Cancer: non-small-cell lung cancer first-line/second and further lines of treatment in advanced disease. Ann Oncol. 2014; 25(8):1475-84.
9. Bjordal et al., “A 12 country field study of the EORTC QLQ-C30 (version 3.0) and the head and neck cancer specific module (EORTC QLQ-H; N35) in head and neck patients. EORTC Quality of Life Group,” Eur J Cancer. 2000; 36(14):1796-807.
10. Borghaei et al., “Nivolumab versus docetaxel in advanced nonsquamous non-small-cell lung cancer,” N Engl J Med. 2015; 373(17):1627-39.
11. Burova E et al. Mol Cancer Ther. 2017; 16:861-870.
12. Carbone et al., “First-Line Nivolumab in Stage IV or Recurrent Non-Small-Cell Lung Cancer,” N Engl J Med. 2017; 276(25):2415-26.
13. Chamberlain M C et al. Neuro Oncol. 2017; 19:i1-i24.
14. Creelan, “Update on immune checkpoint inhibitors in lung cancer,” Cancer Control. 2014; 21(1):80-89.
15. D'Incecco et al., “PD-1 and PD-L1 expression in molecularly selected non-small-cell lung cancer patients,” Br J Cancer. 2015; 112(1):95-102.
16. De Lima Lopes et al., “P2.43: Pembrolizumab vs platinum-based chemotherapy for PD-L1+NSCLC: phase 3, randomized, open-label KEYNOTE-042: track: immunotherapy,” J Thorac Oncol. 2018; 11(10S):S244-S245.
17. Eisenhauer et al., “New response evaluation criteria in solid tumours: revised RECIST guideline (version 1.1),” Eur J Cancer. 2009; 45(2):228-47.
18. Ettinger et al., “NCCN Guidelines insights: non-small cell lung cancer, Version 4.2016,” J Natl Compr Canc Netw. 2016; 14(3):255-64.
19. Fossella et al., “Randomized, multinational, phase III study of docetaxel plus platinum combinations versus vinorelbine plus cisplatin for advanced non-small-cell lung cancer: the TAX 326 study group,” J Clin Oncol. 2003; 21:3016-3024.
20. Gadgeel et al., “Pembrolizumab (pembro) plus chemotherapy as front-line therapy for advanced NSCLC: KEYNOTE-021 cohorts A-C,” J Clin Oncol. 2016; 34(Suppl); abstract 9016.
21. Gandhi et al., “Pembrolizumab plus Chemotherapy in Metastatic Non-Small-Cell Lung Cancer,” N Engl J Med. 2018; 278(22):2078-92.
22. Garon et al., “Pembrolizumab for the treatment of non-small-cell lung cancer,” N Engl J Med. 2015; 372 (21): 2018-28.
23. Gettinger et al., “First-line monotherapy with nivolumab (NIVO; anti-programmed death-1 [PD-1]) in advanced non-small cell lung cancer (NSCLC): safety, efficacy and correlation of outcomes with PD-1 ligand (PD-L1) expression,” J Clin Oncol. 2015; 33(Suppl); abstract 8025.
24. Gettinger et al., “Nivolumab onotherapy for first-line treatment of advanced non-small-cell lung cancer,” J Clin Oncol. 2016; 34(25):2980-87.
25. Goldberg S B et al. Lancet Oncol. 2020; 21:655-663.
26. Goldberg S B et al. Lancet Oncol. 2016; 17:976-983.
27. Iyer S et al. Support Care Cancer. 2014; 22:181-187.
28. Jotte et al., “IMpower131: Primary PFS and safety analysis of a randomized phase III study of atezolizumab+carboplatin+paclitaxel or nab-paclitaxel vs carboplatin+nab-paclitaxel as 1L therapy in advanced squamous NSCLC,” Journal of Clinical Oncology 2018; 36 (18_suppl): LBA9000-LBA.
29. Kerr et al., IASLC Pathology Committee. Programmed death-ligand 1 immunohistochemistry in lung cancer: in what state is this art?” J Thorac Oncol. 2015; 10(7):985-89.
30. KEYTRUDA [package insert]. Whitehouse Station, NY: Merck & Co, 2020.
31. Lopes et al., “Pembrolizumab (pembro) versus platinum-based chemotherapy (chemo) as first-line therapy for advanced/metastatic NSCLC with a PD-L1 tumor proportion score (TPS) ≥1%: Open-label, phase 3 KEYNOTE-042 study,” J Clin Oncol. 2018; 36(18_suppl):LBA4-LBA.
32 Macdonald L E et al. Proc Natl Acad Sci USA. 2014; 111:5147-5152.
33. Murphy A J et al. Proc Natl Acad Sci USA. 2014; 111:5153-5158.
34. National Comprehensive Cancer Network, NCCN Clinical Practice Guidelines in Oncology: Non-small
35. Cell Lung Cancer. Version 4.2021, 2021. Available at: https://www.nccn.org/professionals/physician_gls/pdf/nscl.pdf. Accessed: Apr. 17, 2021.
36. LIBTAYOR [cemiplimab-rwlc] injection full US prescribing information. Available from: https://www.accessdata.fda.gov/drugsatfda_docs/label/2021/761097s007lbl.pdf (2021).
37. OPDIVO [package insert]. Princeton, NJ: Bristol-Myers Squibb Company; 2020.
38. Osoba D et al. J Clin Oncol. 1998; 16:139-144.
39. Paz-Ares et al., “PARAMOUNT: Final overall survival results of the phase III study of maintenance pemetrexed versus placebo immediately after induction treatment with pemetrexed plus cisplatin for advanced nonsquamous non-small-cell lung cancer,” J Clin Oncol. 2013; 31 (23): 2895-902.
40. Paz-Ares et al., “Pembrolizumab plus Chemotherapy for Squamous Non-Small-Cell Lung Cancer,” N Engl J Med. 2018; 379 (21): 2040-51.
41. Reck et al., “ESMO Guidelines Working Group. Metastatic non-small-cell lung cancer (NSCLC): ESMO Clinical Practice Guidelines for diagnosis, treatment and follow-up,” Ann Oncol. 2014; 25 Suppl 3: iii27-39.
42. Reck et al., “Metastatic non-small-cell lung cancer (NSCLC): ESMO Clinical Practice Guidelines for diagnosis, treatment and follow-up,” Ann Oncol 2014; 25 Suppl 3(iii27-39.
43. Reck et al., “Pembrolizumab versus chemotherapy for PD-L1-positive non-small-cell lung cancer,” N Engl J Med. 2016 Oct. 8.
44. Reck et al., “Updated Analysis of KEYNOTE-024: Pembrolizumab Versus Platinum-Based Chemotherapy for Advanced Non-Small-Cell Lung Cancer With PD-L1 Tumor Proportion Score of 50% or Greater,” J Clin Oncol 2019; 37(7):537-46.
45. Robins et al., “Correcting for non-compliance in randomized trials using rank preserving structural failure time models,” Communications in Statistics—Theory and Methods 1991; 20(8):2609-31.
46. Scagliotti et al., “Phase III randomized trial comparing three platinum-based doublets in advanced non-small-cell lung cancer,” J Clin Oncol. 2002; 20(21):4285-91.
47. Schiller et al., “Comparison of four chemotherapy regimens for advanced non-small-cell lung cancer,” N Engl J Med. 2002; 346(2):92-98.
48. Sezer A et al. Lancet. 2021; 397:592-604.
49. Sezer A et al. Ann Oncol. 2020; 31(Suppl 4):S1182-1183.
50. Siegel et al., “Cancer statistics, 2016,” CA Cancer J Clin. 2016; 66:7-30.
51. Socinski et al., “CheckMate 026: A phase 3 trial of nivolumab vs investigator's choice (IC) of platinum-based doublet chemotherapy (PT-DC) as first-line therapy for stage IV/recurrent programmed death ligand 1 (PD-L1)-positive NSCLC,” Annals of Oncology. 2016; 27 (supplement 6): LBA7_PR.
52. TECENTRIQ® [atezolizumab] [package insert]. 2018. In: Genentech, Inc., South San Francisco, CA.
53. Vinay et al., “Immune evasion in cancer: mechanistic basis and therapeutic strategies,” Semin Cancer Biol. 2015; 35:S185-98.
54. Waqar S N et al. Clin Lung Cancer. 2018; 19:e373-e379.
55. Weick et al., “A randomized trial of five cisplatin-containing treatments in patients with metastatic non-small-cell lung cancer: a Southwest Oncology Group study,” J Clin Oncol. 1991: 9(7):1157-62.
56. Whisenant M S et al. JCO Oncol Pract. 2020; 16:e1151-e1160.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method of treating or inhibiting the growth of a tumor, comprising:
(a) selecting a patient with lung cancer and brain metastasis; and
(b) administering to the patient a therapeutically effective amount of a programmed death-1 (PD-1) inhibitor, wherein the PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2, or a bioequivalent thereof.

2. The method according to claim 1, wherein the lung cancer is non-small cell lung cancer.

3. The method according to claim 1, wherein the lung cancer is locally advanced or metastatic non-small cell lung cancer.

4. The method according to claim 1, wherein the lung cancer is locally advanced non-small cell lung cancer.

5. The method according to claim 4, wherein the patient is not a candidate for surgical resection or definitive chemoradiation.

6. The method according to claim 1, wherein the lung cancer is metastatic.

7. The method according to claim 1, wherein the patient has squamous or non-squamous lung cancer.

8. The method according to claim 1, wherein tumor tissue in the patient expresses PD-L1 in ≥50% of tumor cells.

9. The method according to claim 1, wherein the brain metastasis is treated, stable brain metastasis.

10. The method according to claim 1, wherein the patient has no EGFR, ALK or ROS1 aberrations.

11. The method according to claim 1, wherein the anti-PD-1 antibody comprises HCDR1 having an amino acid sequence of SEQ ID NO: 3; HCDR2 having an amino acid sequence of SEQ ID NO: 4; HCDR3 having an amino acid sequence of SEQ ID NO: 5; LCDR1 having an amino acid sequence of SEQ ID NO: 6; LCDR2 having an amino acid sequence of SEQ ID NO: 7; and LCDR3 having an amino acid sequence of SEQ ID NO: 8.

12. The method according to claim 1, wherein the anti-PD-1 antibody comprises a HCVR comprising an amino acid sequence of SEQ ID NO: 1.

13. The method according to claim 1, wherein the anti-PD-1 antibody comprises a LCVR comprising an amino acid sequence of SEQ ID NO: 2.

14. The method according to claim 1, wherein the anti-PD-1 antibody comprises a HCVR/LCVR amino acid sequence pair of SEQ ID NOs: 1/2.

15. The method according to claim 1, wherein the anti-PD-1 antibody comprises a heavy chain and a light chain, wherein the heavy chain has an amino acid sequence of SEQ ID NO: 9.

16. The method according to claim 1, wherein the anti-PD-1 antibody comprises a heavy chain and a light chain, wherein the light chain has an amino acid sequence of SEQ ID NO: 10.

17. The method according to claim 1, wherein the anti-PD-1 antibody comprises a heavy chain and a light chain, wherein the heavy chain has an amino acid sequence of SEQ ID NO: 9 and the light chain has an amino acid sequence of SEQ ID NO: 10.

18. The method according to claim 1, wherein the anti-PD-1 antibody is cemiplimab.

19. The method according to claim 11, wherein the PD-1 inhibitor is an anti-PD-1 antibody comprising a HCVR with at least 90% sequence identity to SEQ ID NO: 1.

20. The method according to claim 11, wherein the PD-1 inhibitor is an anti-PD-1 antibody comprising a LCVR with at least 90% sequence identity to SEQ ID NO: 2.

21. The method according to claim 11, wherein the PD-1 inhibitor is an anti-PD-1 antibody comprising a HCVR with at least 90% sequence identity to SEQ ID NO: 1, and a LCVR with at least 90% sequence identity to SEQ ID NO: 2.

22. The method according to claim 1, wherein the method promotes tumor regression, reduces tumor cell load, reduces tumor burden, and/or prevents tumor recurrence in the patient.

23. The method according to claim 1, wherein the method leads to at least one effect selected from increase in progression-free survival, increase in overall survival, complete response, partial response, and stable disease.

24. The method according to claim 1, wherein the method leads to increase in at least one of progression-free survival, overall survival, and objective response rate, as compared to chemotherapy.

25. The method according to claim 1, wherein the method leads to improved functioning and quality of life of the patient, as measured by EORTC QLQ-C30 and QLQ-LC13, as compared to a patient treated with chemotherapy alone.

26. The method according to claim 1, wherein the method delays the time to definitive deterioration in GHS/QOL of the patient, as measured by EORTC QLQ-C30 and QLQ-LC13, as compared to a patient treated with chemotherapy alone.

27. The method according to claim 1, further comprising administering to the patient an additional therapeutic agent or therapy selected from one or more of: surgery, radiation, an anti-viral therapy, photodynamic therapy, a programmed death ligand 1 (PD-L1) inhibitor, a lymphocyte activation gene 3 (LAG3) inhibitor, a cytotoxic T-lymphocyte-associated protein 4 (CTLA-4) inhibitor, a glucocorticoid-induced tumor necrosis factor receptor (GITR) agonist, a T-cell immunoglobulin and mucin containing-3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an antagonist of another T-cell co-inhibitor or ligand, a CD20 inhibitor, an indoleamine-2,3-dioxygenase (IDO) inhibitor, a CD28 activator, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an agonist to a co-stimulatory receptor, an antibody to a tumor-specific antigen, a vaccine, an adjuvant to increase antigen presentation, an oncolytic virus, a cytotoxin, a chemotherapeutic agent, platinum-based chemotherapy, a tyrosine kinase inhibitor, an IL-6R inhibitor, an IL-4R inhibitor, an IL-10 inhibitor, a cytokine, an antibody drug conjugate (ADC), chimeric antigen receptor T cells, an anti-inflammatory drug, and a dietary supplement.

28. The method of claim 1, wherein the PD-1 inhibitor is administered as one or more doses, wherein each dose is administered two weeks, three weeks, four weeks, five weeks or six weeks after the immediately preceding dose.

29. The method of claim 1, wherein the PD-1 inhibitor is administered as two or more doses, wherein each dose is administered three weeks after the immediately preceding dose.

30. The method of claim 1, wherein the PD-1 inhibitor is administered at a dose of 5 mg to 800 mg.

31. The method of claim 1, wherein the PD-1 inhibitor is administered at a dose of 200 mg, 250 mg, or 350 mg.

32. The method of claim 1, wherein the PD-1 inhibitor is administered at a dose of 1 mg/kg to 20 mg/kg of the patient's body weight.

33. The method of claim 1, wherein the PD-1 inhibitor is administered at a dose of 1 mg/kg, 3 mg/kg or 10 mg/kg of the patient's body weight.

34. The method according to claim 1, wherein the PD-1 inhibitor is administered intravenously, or subcutaneously.

35. (canceled)

36. A kit comprising a programmed death 1 (PD-1) inhibitor in combination with written instructions for use of a therapeutically effective amount of the PD-1 inhibitor for treating or inhibiting the growth of a tumor in a patient with lung cancer and brain metastasis, wherein the PD-1 inhibitor is an antibody that binds specifically to PD-1 and comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2.
